(19) United States
(12) Patent Application Publication
(10) Pub. No.: US 2021/0335476 A1
(43) Pub. Date: Oct. 28, 2021

(54) FOOD PACKAGING SELECTION AND HANDLING

(71) Applicant: LOVED BY LIMITED, London (GB)

(72) Inventor: Matthew Farrar, (US)

(21) Appl. No.: 17/281,089

(22) PCT Filed: Sep. 27, 2019
(86) PCT No.: PCT/GB2019/052741

§ 371 Date: Mar. 29, 2021

(30) Foreign Application Priority Data
Sep. 28, 2018 (GB) 1815909.5
Sep. 28, 2018 (GB) 1815911.1
Sep. 28, 2018 (GB) 1815912.9
Sep. 28, 2018 (GB) 1815914.5

(51) Int. Cl. G16H 20/60; A47J 39/00; G06Q 10/08; G06K 7/10; G16H 10/60; G16H 50/30; G16H 40/67; G06Q 10/10; G16H 50/70; G06K 19/077; G05B 19/042; G01G 19/414; G01K 13/00; G01K 3/00; A61B 5/00; A61B 5/145; A61B 5/11

(52) U.S. Cl. CPC G16H 20/60; G06K 7/1413; G06Q 10/087; G06K 7/10861; G16H 10/60; G16H 50/30; G16H 40/67; G06Q 10/1093; G16H 50/70; G06K 19/07756; G05B 19/042; G01G 19/4146; G01K 13/00; G01K 3/005; A61B 5/4866; A61B 5/14532; A61B 5/1118; G06K 2007/10504; G06K 7/10297; A47J 39/006

(57) ABSTRACT

Four concepts relating to holding and packaging food, determining the nutritional content of food packaging and selecting foodstuffs for consumption by a user. A food holder carries multiple foodstuffs to be stored at different environmental conditions in separate regions, the food holder being adapted to afford communication between each region and an external environment for controlling the environment of each region. Food packaging comprises a tag configured to transmit a signal indicative of the preparation of the food packaging for consumption of food contained in the food packaging. A system determines the nutritional content of food packaging by receiving an identifier associated with food packaging and accessing data associated with the identifier, the data comprising an indication of the type of food stored in the food packaging and a parameter relating to the food stored in the food packaging. Selecting foodstuffs for consumption by a user utilises a method comprising: receiving user data relating to the user, accessing food data relating to a group of food items, determining a predicted physiological effect on the user of each food item of the group of food items in dependence on the food data and the user data, and selecting, in dependence on the predicted physiological effect on the user, one or more food item of the group of food items for consumption by the user.

The present invention relates to four concepts. These are:

Concept A: Food Holder

Concept B: Determining Nutritional Content of Food Packaging

Concept C: Selecting Foodstuffs

Concept D: Food Packaging

### Concept A: Food Holder

### Background

This concept relates to a food holder for holding foodstuffs. In particular, the present invention of concept A relates to a food holder for holding foodstuffs which are to be stored at different environmental conditions.

Bags for holding or carrying food items are known. Such bags can usefully hold multiple different foodstuffs. These bags can be useful for carrying multiple different foodstuffs for relatively short periods of time, for example between a shop and a user's home as a user returns from a shopping trip. However, they are generally unsuitable for longer-term storage of these different foodstuffs. The food items will generally need to be removed from the bag and stored in different parts of a user's kitchen, as appropriate to the food item.

### Summary

According to an aspect of the present invention of concept A, there is provided a food holder for carrying multiple foodstuffs to be stored at different environmental conditions, the food holder comprising: a first region for receiving a foodstuff and a second region for receiving another foodstuff, the second region being at least partly environmentally isolated from the first region, the food holder being adapted to afford communication between the first region and an environment external to the food holder thereby to control an environment within the first region separately from an environment within the second region.

The food holder may comprise a transmissive portion configured to permit the first region to communicate with the external environment. The transmissive portion may form at least part of one or more of an upper, side and lower wall of the first region of the food holder. The transmissive portion may be configured to transmit fluid. The transmissive portion may comprise one or more aperture.

The food holder may comprise one transmissive portion comprising a first aperture in one of the upper, side and lower walls of the first region and another transmissive portion comprising a second aperture in another of the upper, side and lower walls of the first region, thereby creating a fluid flow path through the first region between the first aperture and the second aperture. The first aperture may be a different size to the second aperture.

The food holder may be configured to receive a fluid flow from the external environment and to couple the fluid flow into the fluid flow path through the first region. The transmissive portion may be configured to transmit heat more readily than a remainder of the food holder, thereby to thermally equalise the environment within the first region with that of the external environment.

The food holder may comprise a sensor for sensing an environmental condition within one or both of the first region and the second region. The sensor may comprise a first sensor for sensing the environmental condition within the first region and a second sensor for sensing the environmental condition within the second region. The sensor may comprise a weight sensor for sensing the weight of foodstuffs within one or both of the first region and the second region.

The food holder may comprise an indicator display configured to receive a signal from the sensor and in dependence on the received signal to indicate on the display one or more of: an environmental condition within the first and/or second region, and a weight of a foodstuff within the first and/or second region.

The food holder may comprise a processor coupled to the sensor and to the display, the processor being configured to: determine an estimated expiry date of a foodstuff within the first and/or second region in dependence on sensor data received from the sensor, and cause the display to indicate the estimated expiry date.

According to another aspect of the present invention of concept A there is provided a storage unit adapted to receive a food holder according to any preceding claim for providing separate environmental control over different regions within the food holder, the storage unit comprising: an environmental control mechanism for communicating with a first region of a food holder received by the storage unit thereby to control an environment within the first region separately from an environment within a second region of the food holder.

The environmental control mechanism may comprise a fluid outlet configured to communicate with an aperture of the food holder thereby to couple a fluid flow between the fluid outlet and an interior of the holder. The environmental control mechanism may comprise a fluid inlet configured to communicate with an interior of the food holder thereby to provide a return fluid flow path for fluid flow from the fluid outlet.

The fluid outlet of the environmental control mechanism may be configured to communicate with a first aperture of the food holder and the fluid inlet of the environmental control mechanism may be configured to communicate with a second aperture of the food holder, thereby to couple a fluid flow between the fluid outlet and the fluid inlet through the food holder.

One or both of the fluid outlet and the fluid inlet may be configured for sealing engagement with respective portions of the food holder.

The storage unit may be configured to control one or more of temperature, humidity and flow rate of fluid emitted from the fluid outlet.

The storage unit may comprise a first zone and a second zone, the storage unit being configured to receive the food holder such that the first region of the food holder is received into the first zone and the second region of the food holder is received into the second zone, the storage unit being configured to hold each of the first zone and the second zone at different temperatures thereby to control the temperature of the first region to be different to that of the second region. The first zone may be chilled compared to an ambient room temperature and the second zone may be permitted to remain at the ambient room temperature.

The storage unit may comprise a reader configured to read an identifier associated with the food holder, the storage unit being configured to control the environment within the first region in dependence on the identifier.

The storage unit may be configured to generate an indicator signal thereby to cause an indicator to display an indication of the controlled environment.

According to another aspect of the present invention of concept A there is provided a food storage system for storing a plurality of foodstuffs at different environmental conditions, the food storage system comprising: a food holder as defined herein, and a storage unit as defined herein.

### Concept B: Determining Nutritional Content of Food Packaging

### Background

The present invention of concept B relates to determining nutritional content of food, in particular to determining nutritional content of food contained in food packaging.

Systems exist for finding out the contents of a packaged piece of food. For example, a barcode can be read, and the food item identified from that barcode. Often, however, the identification of the food item is broad, for example indicating a whole number of food items in the packaged food, such as one lettuce or six tomatoes.

It is desirable to be able to determine the contents of food packaging with a greater accuracy.

### Summary

According to an aspect of the present invention of concept B there is provided a system for determining the nutritional content of food packaging, the system comprising: a communication module configured to receive an identifier associated with food packaging, and a processing module configured to: access, in dependence on the received identifier, data relating to the food packaging associated with the identifier, the data being stored at a memory, the data comprising: an indication of the type of food stored in the food packaging, and a parameter relating to the food stored in the food packaging determined on packaging the food packaging; and determine, in dependence on the accessed parameter, the nutritional content of the food packaging identified by the identifier.

The parameter may comprise one or more of: a weight of the food in the food packaging, a volume of the food in the food packaging, an indication of the atmosphere in which the food in the food packaging was packaged, and a date on which the food in the food packaging was packaged. The communication module may be configured to receive an indication of the temperature profile of the food packaging over time and the processing module may be configured to determine the nutritional content of the food packaging in dependence on the received temperature profile. The system may further be configured to output a signal indicating the nutritional content of the food packaging.

The communication module may be configured to receive a plurality of identifiers, each identifier being associated with respective food packaging and the processing module may be configured, in dependence on the respective identifier, to access data relating to the respective food packaging and determine the nutritional content of each of the plurality of food packaging.

The system may further be configured to output a signal indicating the nutritional content of the plurality of food packaging.

The system may comprise a reader for reading the identifier or the plurality of identifiers, the reader being configured to send the identifier or the plurality of identifiers to the communication module. The reader may be remote from the memory. The reader may be remote from the communication module. The memory may be provided at a server remote from the reader.

According to another aspect of the present invention of concept B there is provided a method of determining the nutritional content of food packaging, the method comprising: receiving an identifier associated with food packaging, accessing, in dependence on the received identifier, data relating to the food packaging associated with the identifier, the data comprising an indication of the type of food stored in the food packaging, and a parameter relating to the food stored in the food packaging determined on packaging, and determining, in dependence on the accessed parameter, the nutritional content of the food packaging identified by the identifier.

The parameter may comprise one or more of: a weight of the food in the food packaging on packing, a volume of the food in the food packaging on packing, an indication of the atmosphere in which the food in the food packaging was packaged, and a date on which the food in the food packaging was packaged.

The method may comprise receiving an indication of the temperature profile of the food packaging over time and determining the nutritional content of the food packaging in dependence on the received temperature profile. The method may comprise outputting a signal indicating the nutritional content of the food packaging. The method may comprise receiving a plurality of identifiers, each identifier being associated with respective food packaging and, in dependence on the respective identifier, accessing data relating to the respective food packaging and determining the nutritional content of each of the plurality of food packaging.

The method may comprise outputting a signal indicating the nutritional content of the plurality of food packaging. The method may comprise reading the identifier or the plurality of identifiers.gb

### Concept C: Selecting Foodstuffs

### Background

The present invention of concept C relates to selecting foodstuffs. In particular, the present invention of concept C relates to selecting foodstuffs from a group of foodstuffs for consumption by a user. For example, the foodstuffs can be provided in food packaging.

Users typically select food for consumption in one of two approaches. In a first approach, the user can decide what meal they would like to eat, and can then select the ingredients for that meal from stored ingredients. In another approach, the user can see what ingredients they have and can decide what meal to make based on those ingredients.

Users can choose to have relatively healthier meals, such as fish and vegetables, or relatively unhealthier meals, such as burgers and chips. Also, a particular foodstuff can be cooked in different ways which can alter the calorific content of that foodstuff. For example food can be grilled or deep fried. In this way, users can, to some extent, control calorific intake.

It is desirable for there to be more accurate control over the food consumed by a user.

### Summary

According to an aspect of the present invention of concept C there is provided a method for recommending a food item from a group of food items for consumption by a user, the method comprising: receiving user data relating to the user, accessing food data relating to the group of food items, the food data comprising nutritional content of each of the food items in the group of food items, determining a predicted physiological effect on the user of each food item of the group of food items in dependence on the food data and the user data, and selecting, in dependence on the predicted physiological effect on the user, one or more food item of the group of food items for consumption by the user.

The user data may comprise one or more of: physiological data relating to the user, activity data relating to the user, and data relating to food consumed by the user within a predetermined period of time preceding the selection of the food item for consumption by the user.

The physiological data relating to the user may comprise one or more of: a height and/or weight of the user, a fitness level of the user, at least a portion of a DNA profile of the user, a blood sugar level of the user, a metabolic state of the user, and food consumption history of the user.

The physiological data may comprise data based on one or more of current physiological data and previous physiological data.

The method may comprise determining the blood sugar level of the user in dependence on a signal from a blood sugar monitor.

The activity data relating to the user may comprise one or more of: a current activity state of the user, a previous activity state of the user, and an expected activity state of the user. The activity data may be determined in dependence on one or more of: manually input data relating to at least one of a current activity, a previous activity and an expected activity, a tracking device, and a calendar entry.

The method may comprise determining that a change in the user's physiological data has occurred which indicates that an activity has been undertaken by the user, requesting user input relating to the indicated activity, and storing one or both of the day and time of day together with the user input, and subsequently determining the activity data in dependence on the stored data.

The method may comprise monitoring the user's physiological response to a particular food item, and selecting the food item for consumption by the user in dependence on the user's previous physiological response to that particular food item.

The selection of the one or more food item of the group of food items for consumption by the user may comprise determining a proportion of a food item for consumption by the user.

The method may comprise generating a signal indicative of the selected one or more food item of the group of food items for consumption by the user, thereby to cause an indicator to indicate the selected one or more food item. The method may comprise receiving the signal at a food holder containing at least one food item and causing an indicator display of the food holder to indicate the selected one or more food item. The method may comprise receiving the signal at a mobile device and causing an indicator at the mobile device to indicate the selected food item.

According to another aspect of the present invention of concept C there is provided a system for recommending a food item from a group of food items for consumption by a user, the system comprising a processor configured to: receive user data relating to the user, access food data relating to the group of food items, the food data being stored at a memory accessible to the processor, the food data comprising nutritional content of each of the food items in the group of food items, determine a predicted physiological effect on the user of each food item of the group of food items in dependence on the food data and the user data, and select, in dependence on the predicted physiological effect on the user, one or more food item of the group of food items for consumption by the user.

The user data may comprise one or more of: physiological data relating to the user, activity data relating to the user, and data relating to food consumed by the user within a predetermined period of time preceding the selection of the food item for consumption by the user. The physiological data relating to the user may comprise one or more of: a height and/or weight of the user, a fitness level of the user, at least a portion of a DNA profile of the user, a blood sugar level of the user, a metabolic state of the user, and food consumption history of the user. The physiological data may comprise data based on one or more of current physiological data and previous physiological data. The blood sugar level of the user may be determined in dependence on a blood sugar monitor.

The activity data relating to the user may comprise one or more of: a current activity state of the user, a previous activity state of the user, and an expected activity state of the user. The activity data may be determined in dependence on one or more of: manually input data relating to at least one of a current activity, a previous activity and an expected activity, a tracking device, and a calendar entry.

The processor may further be configured to: determine that a change in the user's physiological data has occurred which indicates that an activity has been undertaken by the user, request user input relating to the indicated activity, store one or both of the day and time of day together with the user input, and determine the activity data in dependence on the stored data.

The processor may further be configured to: monitor the user's physiological response to a particular food item, and select the food item for consumption by the user in dependence on the user's previous physiological response to that particular food item.

The processor may be configured to select the one or more food item of the group of food items for consumption by the user by determining a proportion of a food item for consumption by the user.

The processor may be configured to generate a signal indicative of the selected one or more food item of the group of food items for consumption by the user, thereby to cause the display of the selected one or more food item.

The system may comprise a food holder containing at least one food item, the food holder being configured to receive the signal and cause an indicator display of the food holder to indicate the selected one or more food item. The system may comprise a mobile device configured to receive the signal and cause an indicator at the mobile device to indicate the determined food item.

### Concept D: Food Packaging

### Background

The present invention of concept D relates to food packaging for packaging food. In particular, the present invention of concept D relates to food packaging which can indicate preparation of the food packaging for consumption of food contained in the food packaging.

It is known to provide systems that attempt to track food consumption, for example so as to enable automatic restocking of food supplies. Such systems typically attempt to track food consumption by tracking food containers as they are moved within a kitchen. Such tracking of food containers can be inaccurate, leading to inaccuracies in tracking food consumption.

It is desirable to provide for more accurate determination of food consumption.

### Summary

According to an aspect of the present invention of concept D, there is provided food packaging comprising a tag configured to transmit a signal indicative of the preparation of the food packaging for consumption of food contained in the food packaging.

The tag may be configured to detect an indication of the preparation of the food packaging. The tag may comprise a sensor configured to detect the separation of one portion of the food packaging from another portion of the food packaging and to generate a separation signal in dependence on the detected separation, the signal indicative of the preparation comprising the separation signal.

The tag may be configured to transmit the signal indicative of the preparation in dependence on the detection of the separation. The tag may comprise a proximity sensor configured to detect the separation and to generate the separation signal in dependence on the detected separation. The tag may comprise one or both of a magnetic sensor and a capacitive sensor for detecting the separation.

The food packaging may comprise a cover and a body, and the tag may be configured to detect the separation of a portion of the cover from the body and to generate the separation signal in dependence on determining that the portion of the cover is separated from the body.

The tag may be configured to detect the separation in dependence on determining a change in an electrically conductive path through at least a portion of the food packaging.

The tag may be configured to detect a temperature of the food packaging and to generate a readiness signal in dependence on the temperature, the signal indicative of the preparation comprising the readiness signal. The tag may be configured to transmit the signal indicative of the preparation in dependence on the readiness signal.

The tag may be configured to generate the readiness signal in dependence on the temperature satisfying a temperature criterion. The tag may be configured to detect a temperature profile of the food packaging over time, and to determine that the temperature satisfies the temperature criterion in dependence on the detected temperature profile. The temperature criterion may comprise a threshold temperature, and the tag may be configured to determine that the temperature criterion is satisfied in dependence on the temperature exceeding the threshold temperature.

The temperature criterion may comprise a threshold thermal budget, and the tag may be configured to determine that the temperature criterion is satisfied in dependence on the temperature profile indicating that the thermal budget of the food packaging exceeds the threshold thermal budget.

The temperature criterion may comprise a further threshold temperature, and the tag may be configured to determine that the temperature criterion is satisfied in dependence on one or both of: the temperature exceeding the threshold temperature, and the thermal budget of the food packaging exceeding the threshold thermal budget; and in dependence on the temperature subsequently falling below the further threshold temperature.

The tag may be configured to determine movement of the food packaging and to transmit the signal indicative of the preparation in dependence on the determined movement. The tag may be configured to determine movement of the food packaging in dependence on a weight signal generated by a weight sensor.

The tag may be configured to determine a distance from a location external to the food packaging and to determine movement of the food packaging in dependence on the determined distance. The tag may be configured to determine the distance in dependence on a received signal strength of a signal generated at the external location.

The signal indicative of the preparation of the food packaging may identify the food contained in the food packaging.

According to another aspect of the present invention of concept D there is provided a method for detecting the readiness for use of food packaging, the method comprising: receiving a signal transmitted by a tag associated with the food packaging, the signal indicating preparation of the food packaging for consumption of food contained in the food packaging, and determining readiness for use of the food packaging in dependence on the received signal.

The method may comprise determining, in dependence on the received signal, a separation between one portion of the food packaging and another portion of the food packaging, and determining the readiness for use in dependence on the determined separation.

The method may comprise determining, in dependence on the received signal, a temperature of the food packaging, and determining the readiness for use in dependence on the determined temperature. The method may comprise determining that the temperature exceeds a threshold temperature and determining the readiness for use in dependence thereon.

The method may comprise determining a thermal budget of the food packaging based on the determined temperature; determining that the thermal budget exceeds a threshold thermal budget and determining the readiness for use in dependence thereon.

The method may comprise determining the readiness for use in dependence on: determining that the temperature exceeds a threshold temperature, and/or determining that a thermal budget of the food packaging exceeds a threshold thermal budget; and determining that the temperature subsequently falls below a further threshold temperature.

The method may comprise determining movement of the food packaging and determining readiness for use in dependence on the determined movement. The method may comprise receiving a weight signal generated by a weight sensor and determining movement in dependence thereon. The method may comprise determining a distance between the food packaging and a location external to the food packaging, and determining movement in dependence thereon.

### Concepts A-D

Any one or more feature of any aspect above may be combined with any one or more feature of any other aspect above. Any apparatus feature may be written as a method feature where possible, and vice versa. These have not been written out in full here merely for the sake of brevity.

Summaries are provided to introduce a selection of concepts in a simplified form that are further described below in the Detailed Description. The mention of features in these summaries does not indicate that they are key features or essential features of the invention or of the claimed subject matter, nor is it to be taken as limiting the scope of the claimed subject matter.

### BRIEF DESCRIPTION OF THE DRAWINGS

The present invention will now be described by way of example with reference to the accompanying drawings.

In the drawings:

FIG. 1 illustrates an example of a food holder and a storage unit;

FIGS. 2A to 2E illustrate other examples of food holders;

FIGS. 3A to 3C illustrate examples of interfacing between a storage unit and a food holder;

FIGS. 4A and 4B illustrate another example of a food holder;

FIG. 5 illustrates another example of interfacing between a storage unit and a food holder;

FIG. 6 illustrates a system for determining nutritional content of food packaging;

FIG. 7 illustrates a method for determining nutritional content of food packaging;

FIG. 8 illustrates a method for selecting food packaging;

FIG. 9 illustrates a system for selecting food packaging;

FIG. 10 illustrates an example of food packaging;

FIG. 11 illustrates another example of food packaging; and

FIG. 12 illustrates a method for determining the readiness for consumption of food in food packaging.

### DETAILED DESCRIPTION

The techniques described herein are applicable to food packaging, holding and storage systems, and to uses thereof. The techniques are useful in the delivery and storage of foodstuffs for consumption.

The techniques can be used in situations where knowledge or control over foodstuffs for consumption is desirable. One example of this is where a user would like to follow a particular diet. The present techniques enable the user the obtain accurate information regarding the food consumed, and enable an appropriate selection of food to be consumed that can be tailored to a user's needs. The user's needs can vary with time, for example they can be different on different days and can be different at different times of the same day. A user will typically not be able to manage a complicated needs scenario on their own and so need assistance from systems that can provide additional information and advice to the user.

A user may like to follow a particular diet for various reasons. The user may simply wish to maintain a healthy diet. The user may be an athlete on a training program. It may be important to determine the food consumed, and potentially also the time at which it is consumed, to ensure adherence to the training program.

The user may wish to follow a particular diet for health reasons, for example to control a health issue. Examples include users wishing to control their diet to manage one or more of a diabetic condition, a coeliac condition (a gluten-related health issue) or other form of food intolerance, and other health issues that can be managed through diet, for example ADHD.

In general, one or more of the above circumstances can be encompassed by the term ‘dietary condition’.

As mentioned, an example dietary condition is diabetes. Monitoring a diabetic user's diet can be very important. A diabetic user needs to very carefully monitor their food intake so as to be able to regulate blood sugar levels by administering insulin in a dosage and at a time that is appropriate.

This is difficult to achieve in practice, and users may experience large swings in blood sugar levels as sugar from ingested food enters the blood stream, and is regulated by the later administration of insulin by the user. Such swings in blood sugar level are undesirable, even where the variation may be within preferred upper and lower bounds for the blood sugar level.

At least some of the techniques discussed herein can assist users, for example users with a dietary condition, to more accurately regulate their food intake. This can, for example, help diabetic users to more accurately regulate their blood sugar levels.

### Food Storage System

A food storage system 100 comprises a storage unit 120 and a food holder 130. The storage unit 120 is configured to receive a plurality of food holders 130. The storage unit 120 is configured to communicate with a memory 140. The memory may be remote from the storage unit. The storage unit comprises a communication module for communicating with the memory 140. The communication module is suitably configured to communicate via one or both of a wired link and a wireless link. The communication module is, in some examples, configured to communicate via a wireless protocol such as WiFi and/or Bluetooth. Generally, the communication module is configured to communicate with the memory over radio frequencies.

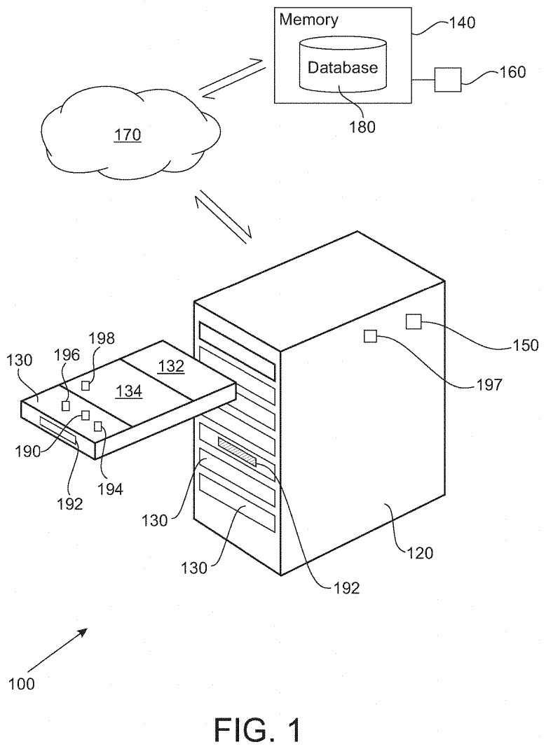

The memory is suitably provided with a further communication module 160 to facilitate communication with the communication module 150 of the storage unit 120. The storage unit 120 and the memory 140 can, in some examples, communicate via the cloud (indicated generally at 170 in FIG. 1). In some examples, the memory 140 can be located in the cloud.

The memory may comprise a database 180 for storing data. The data may be received from the storage unit for storing at the memory. The data may be accessed by the storage unit. Further detail relating to the interaction of the storage unit and the memory will be provided elsewhere herein.

The food holder 130 is schematically shown as comprising different regions, including a first region 132 and a second region 134.

When storing foodstuffs in the food holder, it is useful to be able to store different foodstuffs at different environmental conditions. For example, it will be appropriate to store dry rice at a different environmental condition from that at which raw chicken or vegetables should be stored.

Dry rice can be stored in a relatively dry atmosphere at room temperature (e.g. 25 degrees Celsius). Raw chicken should be chilled to below room temperature, for example to approximately 5 degrees Celsius. Thus the rice and chicken have different environmental storage requirements. It may be desirable to store vegetables in a relatively more humid atmosphere than that at which the rice is stored, and also to chill the vegetables to increase the time for which they can be stored without going off. Thus the rice and vegetables have different environmental storage requirements.

It is known to store rice in a cupboard and to store chicken and vegetables in a fridge. This can, however, be inconvenient as a user will need to unpack items from a shopping trip individually and to put them in a desired location. Further, there may be some foodstuffs for which a user is unsure about the storage requirements, e.g. whether a relatively drier or a relatively more humid atmosphere is most appropriate for that foodstuff. This can lead to the user putting the food item in a less than optimal location, which can affect the useful life of that item.

Further, when a user comes to preparing a meal using the ingredients, for example the rice, chicken and vegetables, the user will need to identify where each of these items is located and to retrieve the items from the respective locations separately. This can be inconvenient. This can be particularly so for a user with limited mobility.

It is therefore desirable to be able to store multiple different foodstuffs in a food holder, but still to provide for each foodstuff to be stored at an appropriate environmental condition for that foodstuff.

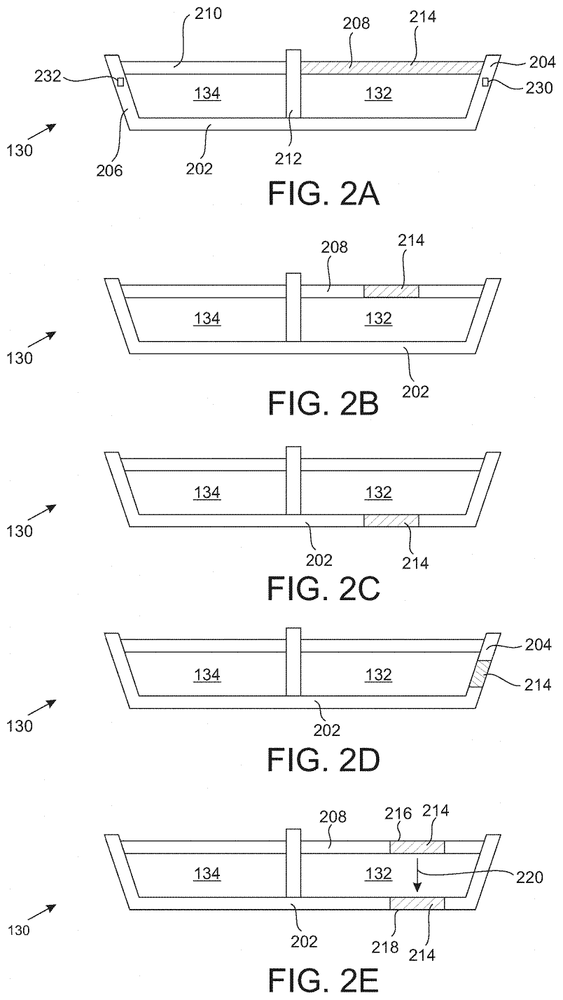

Food Holder Referring now to FIGS. 2A to 2E, a description of examples of the food holder will be provided. It will be understood by the skilled person that the food holder can take many different forms. The examples illustrated in FIGS. 2A to 2E are exemplary. The depictions in FIGS. 2A to 2E are not intended to limit the food holder to holders with a particular shape or size, but they are provided to illustrate embodiments of the holder, as will be apparent from the description which follows.

The food holder 130 comprises a base (or lower wall) 202, side walls 204, 206, and a lid (or upper wall) (illustrated at 208, 210). In the illustrated example a baffle 212 is provided which defines a barrier in the food holder 130, dividing an interior of the food holder into the first region 132 and the second region 134. The baffle 212 provides a flow restriction between the first region and the second region. This arrangement is one way in which an environmental isolation between the first region and the second region can be achieved. As illustrated, the baffle 212 is a solid barrier between the first and second regions of the food holder. The baffle need not be solid in all embodiments. For example, it can be sufficient for the environment within the first region 132 to be partially isolated from the environment within the second region 134, rather than being fully isolated.

Referring again to FIG. 2A, the lid of the food holder 130 is provided in two parts: a first lid 208 which covers the first region 132 and a second lid 210 which covers the second region 134. Thus the food holder 130 comprises separate regions 132, 134 which are at least partially environmentally isolated from one another.

The first region 132 is for receiving a foodstuff. The second region 134 is for receiving another foodstuff. Providing different foodstuffs in each of the first and second regions allows the environmental conditions of each of those regions to be adapted to suit the particular foodstuff held in that region.

The foodstuffs can, in some embodiments be provided directly on the food holder 130, in the first region 132 and in the second region 134. In other embodiments, foodstuffs can be provided in food packaging, and the food packaging can be located on the food holder. For example, food packaging containing one foodstuff can be provided in the first region. Food packaging containing another foodstuff can be provided in the second region.

In the illustrated embodiments, the food holder 130 and the storage unit 120 are each adapted to interface with one another. The interfacing between the food holder 130 and the storage unit 120 affords communication between at least the first region 132 of the food holder 130 and an environment which is external to the food holder, such as an environment inside the storage unit 120. The communication between the first region and the environment inside the storage unit enables the environmental condition within the first region to be controlled by controlling the environment inside the storage unit.

The food holder 130 illustrated in FIG. 2A comprises a transmissive portion 214. The transmissive portion suitably has a transmissivity that is greater than a remaining portion of the food holder. For example, the transmissive portion may allow the transmission of fluid, for example a gas such as air, or heat, more easily than a remaining portion of the food holder, as will be discussed in more detail herein. The transmissive portion 214 is provided at the first region 132. As illustrated in the example of FIG. 2A, the first lid 208 comprises the transmissive portion 214. In this example, the transmissive portion forms the whole of the first lid, but this need not be the case. As illustrated in the example of FIG. 2B, the first lid 208 comprises the transmissive portion 214, but here the transmissive portion is only a part of the first lid.

In another example, illustrated in FIG. 2C, the base 202 comprises the transmissive portion 214. The transmissive portion is provided in a part of the base defining the bottom of the first region. In another example, illustrated in FIG. 2D, a side wall 204 comprises the transmissive portion 214. In FIGS. 2C and 2D the transmissive portion is shown as a part of the base and side wall respectively. In other examples, the transmissive portion can form the whole of the base adjacent the first region and the whole of the side wall adjacent the first region, respectively. More generally, the transmissive portion can form a part or the whole of one or more of the first lid, the base adjacent the first region, and the side wall adjacent the first region.

In general, a portion of the food holder 130 defining a boundary between the first region and the environment external to the food holder comprises the transmissive portion.

The transmissive portion need not be provided solely in one location. As illustrated in FIG. 2E, the food holder comprises a transmissive portion in the first lid 208 and another transmissive portion in the base 202. Other configurations of multiple transmissive portions are possible.

The provision of the transmissive portion means that the communication between the first region and the external environment is different to the communication between the second region and the external environment. This arrangement permits the external environment to have a different influence on the first region compared to the second region. The environmental isolation between the first region and the second region means that the environment within the second region is at least to some extent isolated from the external environment. This arrangement allows the external environment to control the environment in the first region separately from that in the second region.

Suitably the transmissive portion is configured to transmit fluid such as air.

In some examples, a fluid flow such as an airflow can be provided by the storage unit to the food holder to maintain a temperature in the first region of the food holder or to change a temperature in the first region of the food holder. For example, a cooling airflow provided to the first region is not passed to the second region, because of the environmental isolation between the regions.

Thus, the first region will be cooled to a temperature lower than that of the second region. In some examples, a cooling airflow can be passed to the first and second regions at different flow rates. For example, where the first and second regions are not fully environmentally isolated from one another, a relatively small airflow can be provided to the second region compared to the airflow provided to the first region. In this example as well, first and second regions can be maintained at different temperatures.

The transmissive portion comprises, in some examples, one or more aperture. The one or more aperture may be provided with an air grill or vent for directing airflow through the aperture. The transmissive portion can comprise a fluid-permeable cover such as a membrane, for example a fabric such as gauze. The fabric may be a natural fabric or it may be an artificial fabric.

Referring to FIG. 2E, in some examples the food holder has one transmissive portion comprising a first aperture 216 in the first lid of the first region and another transmissive portion comprising a second aperture 218 in the base of the first region, thereby creating a fluid flow path 220 through the first region 132 between the first aperture and the second aperture. The apertures need not be of the same size.

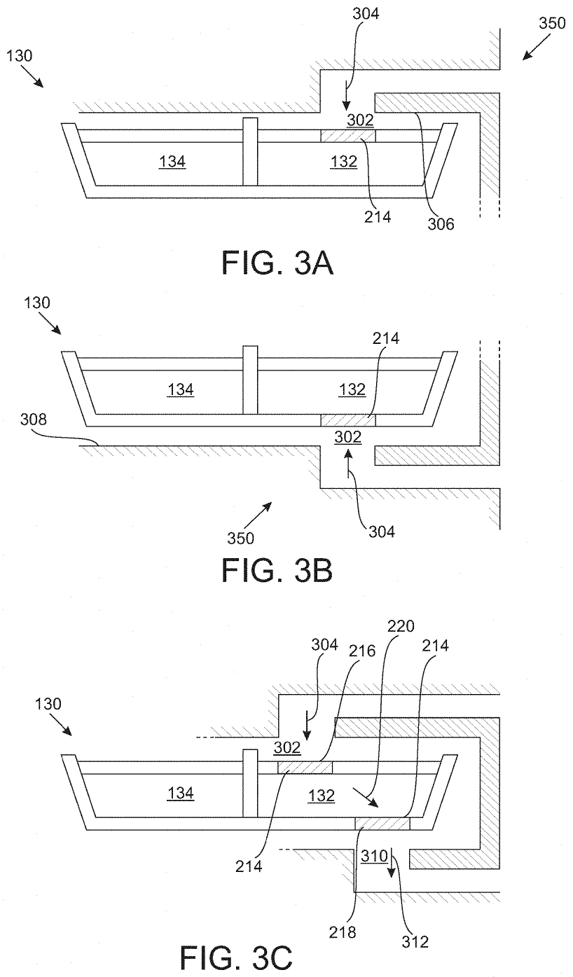

Reference is now made to FIGS. 3A to 3C to illustrate an example of how the storage unit 120 can effect environmental control by coupling a fluid flow into the first region 132. When properly located in the storage unit, the food holder can be located such that the transmissive portion, e.g. an aperture, of the first region is adjacent a fluid outlet 302 of the storage unit. The storage unit can be configured to cause fluid to flow out of the outlet 302 towards the food holder, as indicated at 304. The fluid outlet 302 can be provided towards or as part of a ceiling 306 in the storage unit, as illustrated in FIG. 3A or towards or as part of a floor 308 in the storage unit, as illustrated in FIG. 3B.

In examples where the food holder 130 comprises a transmissive portion in the first lid and a transmissive portion in the base, the food holder can be located such that, when the food holder is properly located in the storage unit, one of the transmissive portions aligns with the fluid outlet 302 and the other of the transmissive portions aligns with a fluid inlet 310 of the storage unit. The storage unit can be configured to accept fluid flow through the fluid inlet 310, as indicated at 312. The fluid flow can be driven by positive fluid pressure from the fluid outlet, by drawing fluid through the fluid inlet, ora combination of these approaches.

As illustrated in FIG. 3C, the fluid outlet 302 is provided in a ceiling 306 of the storage unit and the fluid inlet 310 is provided in a floor 308 of the storage unit.

In some examples, the transmissive portion is configured to transmit heat more readily than a remainder of the food holder, thereby permitting quicker thermal transfer between the environment external to the food holder and the environment within the first region. This can enable thermal equalisation between the environment within the first region and that of the external environment.

The food holder comprises an interior wall or baffle, separating the first region and the second region. The interior wall is suitably of a material with a lower transmissivity than the transmissive portion. For example, where the transmissive portion allows the transmission of fluid such as air, the interior wall may be a solid wall which restricts the passage of fluid. In some examples the interior wall may be waterproof. The first region may be separated from the second region by a fluid-impermeable barrier. In some examples, where the transmissive portion allows the transmission of heat, the interior wall may be of a material with a lower thermal conductivity than the transmissive portion. For example, the transmissive portion may be or may comprise a metallic material, such as by being constructed from metal foil, and the interior wall may be a relatively thermally insulating material such as cardboard, plastic and the like. One or more wall of the food holder may be thermally insulated. This construction permits the maintenance of different thermal environments at each of the first and second regions of the food holder. For example, the first region may be chilled below ambient room temperature and the second region may remain at ambient room temperature.

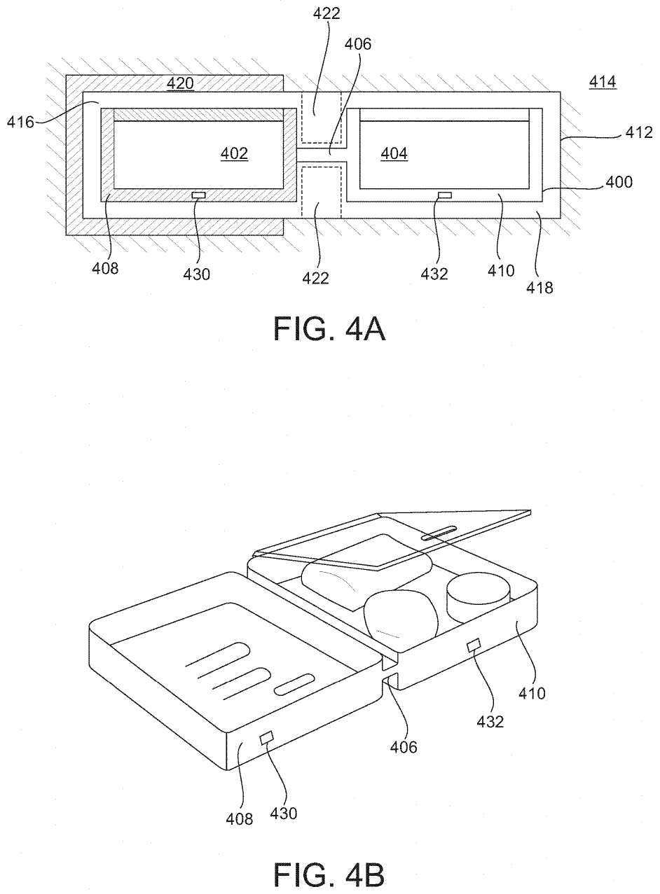

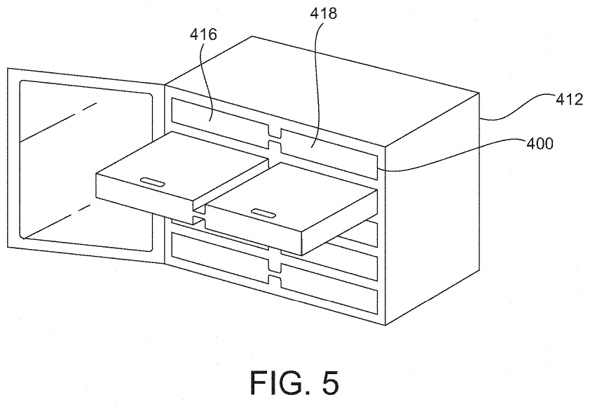

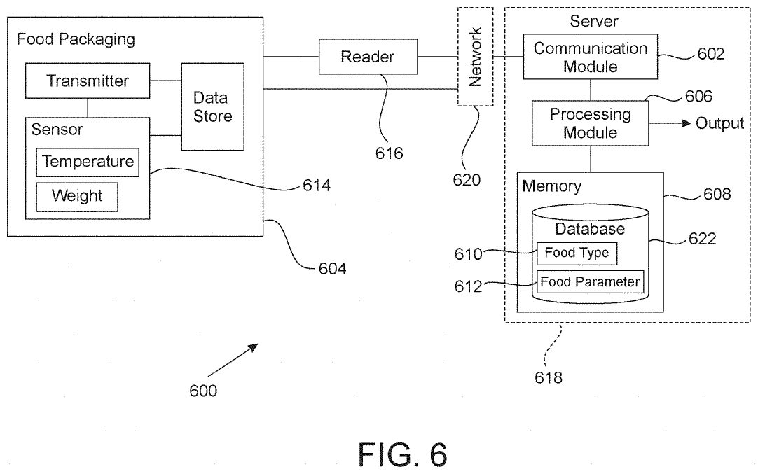

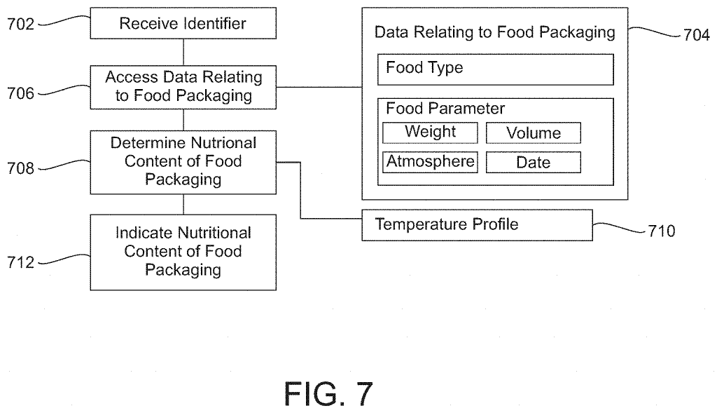

An alternative configuration of a food holder is illustrated in FIGS. 4A and 4B. FIG. 5 illustrates the food holder interfacing with a storage unit. The food holder 400 comprises a first region 402 and a second region 404 separated by a connecting region 406. The walls defining the first region define a thermally transmissive portion. The thermally transmissive portion 408 is more thermally conductive than either of the connecting region 406 and walls defining the second region 410. This configuration allows a faster thermal transfer between an external environment and the first region than between an external environment and the second region.

The food holder 400 is receivable in a generally slot-shaped aperture 412 in the storage unit 414. The aperture in the storage unit defines a plurality of zones: a first zone 416 (towards the left in FIG. 4A) and a second zone 418 (towards the right in FIG. 4A). The storage unit can be configured to maintain each of the zones at different temperatures. This can be achieved by, for example, providing a cooling element adjacent at least part of the walls of the first zone 416. The thermal gradient between the first zone 416 and the second zone 418 can be enhanced or maintained by the provision of baffles 422. The food holder 400 is adapted to cooperate with the storage unit by having a narrowed connecting region 406. The connecting region 406 defines recesses in the upper and lower surfaces of the food holder 400. The baffles 422 can protrude into the recesses. This arrangement can cause a labyrinthine flow path across the food holder from the first region towards the second region, which can restrict the flow of fluid, thereby helping to maintain the temperature gradient between the first zone and the second zone.

The thermally transmissive portion of the food holder allows the cooler temperature of the first zone to cool the environment within the first region. The relatively thermally insulating portion of the food holder defining the second region restricts the transmission of thermal energy out of the second region. Thus the first region can be maintained at a lower temperature than the second region.

The food holder 130 can, in some examples, comprise a sensor (shown schematically at 190 in FIG. 1) for sensing an environmental condition within the food holder, for example within one or both of the first region and the second region. Providing the food holder with a sensor enables the sensing of the environmental condition within the food holder and/or within respective regions of the food holder.

The sensor comprises a first sensor 230, 430 for sensing the environmental condition within the first portion 132, 402 and a second sensor 232, 432 for sensing the environmental condition within the second portion 134, 404. (Note that the first and second sensor are indicated in FIG. 2A. Such sensors are not shown in FIGS. 2B to 2E, but it will be understood that such sensors may be provided in these examples if desired.) Providing respective sensors for each region of the food holder can enable a more accurate determination of the environmental conditions within those regions. In some examples, the first region comprises the first sensor 230, 430 and the second region comprises the second sensor 232, 432. The first sensor may be embedded within or applied to a wall defining a part of the first region. The second sensor may be embedded within or applied to a wall defining a part of the second region. Providing the respective sensors at each region of the food holder can enable the determination of the environmental condition within the respective region to more accurately reflect temporal changes in the environmental condition, without needing to wait for an equilibrium to be established throughout the respective regions of the food holder.

The sensor can, in some examples, comprises a weight sensor for sensing the weight of foodstuffs within one or both of the first region and the second region. This can provide more accurate information relating to the stored foodstuffs.

In some examples, the sensor is configured to sense the location of foodstuffs in the food holder. In some examples, the sensor can comprise a matrix of sensors such as weight or pressure sensors. The matrix of sensors can be provided on an interior floor of the food holder. Where a foodstuff is present in the food holder, it will activate the sensors in the matrix of sensors on which it rests. This permits determination of the presence of the foodstuff. Further, the extent of the activation of the matrix of sensors enables the size of the foodstuff to be determined. For example, where a foodstuff occupies an area of 5 cm×5 cm, a corresponding 5 cm×5 cm area of sensors in the matrix of sensors may be activated. This can assist with the environmental control of the regions of the food holder because the size and volume of the foodstuff can advantageously be taken into account when providing the environmental control, for example the airflow. For example a greater airflow can be provided for cooling a larger volume foodstuff. The location of the activated sensors in the matrix of sensors can enable an automatic determination of the region of the food holder in which the foodstuff is stored. Where more than one foodstuff is stored in the food holder, the matrix of sensors can indicate which foodstuff is stored in which region, for example by comparing the weight or extent of the foodstuffs. Suitably the storage unit is configured to control the environment within the food holder in dependence on the location of foodstuffs in the food holder.

In some examples, the food holder comprises an indicator display 192 configured to receive a signal from the sensor 190 and in dependence on the received signal to indicate on the display one or more of an environmental condition within the first and/or second region, and a weight of a foodstuff within the first and/or second region.

The indicator display can form part of an exposed face of the food holder when received into the storage unit so that the indicator can be seen by a user. A further indicator display may be provided that is separate from the food holder. Such a further indicator display may be provided instead of or as well as the indicator display of the food holder. For example, a user device, such as a portable electronic device, for example a mobile telephone, a tablet computer or a laptop computer, can be configured to receive the indicator signal and display the indication on a screen thereof.

In some examples, the food holder comprises a processor 194 coupled to the sensor 190 and to the display 192, the processor being configured to determine an estimated expiry date of a foodstuff within the first and/or second region in dependence on sensor data received from the sensor, and cause the display to indicate the estimated expiry date.

Suitably the processor is configured to estimate the expiry date of the foodstuff in dependence on the temperature at which the foodstuff is held. The processor may have access to a memory, for example a memory 196 at the food holder and/or a memory 140 in or accessible through the cloud 170. The processor may be configured to store sensor data at the memory. In this way, the processor can access historical sensor data. A temperature profile of the first and/or second region can be used to increase the accuracy of the expiry date determination. A weight, or a weight profile, of a foodstuff in the first and/or second region can also be stored in the memory. The weight or weight profile can be used to increase the accuracy of the expiry date determination.

The sensor 190 can sense environmental conditions within the food holder 130 during transport between a packaging site and a user's location. Additionally or alternatively, the first sensor 230, 430 and/or the second sensor 232, 432 can sense respective environmental conditions within the first and second regions of the food holder during transport between a packaging site and a user's location. This permits the storage conditions during transport to be taken into account when determining the environmental conditions at which to store a foodstuff, and/or when determining the estimated expiry date of the foodstuff in the food holder.

For example, where it is determined that the temperature of the foodstuff or food holder exceeded a threshold temperature for a part of the transport (or indeed, at any time; the time of transportation is being used here merely as an example), the expiry date can be determined to be sooner than would otherwise have been the case. Thus the present approach permits a more accurate determination of the expiry date of a foodstuff, in dependence on the particular conditions at which that foodstuff has been stored. This can provide a greater accuracy than basing a determination on an estimation of the conditions at which a foodstuff is stored.

The determined expiry date can be caused to be displayed on the indicator display and/or on the further indicator display.

### Storage Unit

The above discussion focussed on the food holder for use with the storage unit. In the following, detail will be provided relating to the storage unit. The storage unit 120 is suitably adapted to receive the food holder 130 for providing separate environmental control over different regions within the food holder. The storage unit comprises an environmental control mechanism for communicating with a first region of the food holder thereby to control an environment within the first region separately from an environment within a second region of the food holder. The environmental control mechanism is illustrated schematically at 350 in FIGS. 3A and 3B.

Suitably the storage unit is configured to receive a plurality of food holders. For example the storage unit can be configured to receive at least 5 food holders, and preferably at least 7 food holders. Suitably the storage unit is configured to cooperate with the food holder, or food holders, thereby to control the environmental condition within the first region of a respective food holder.

In some examples, the environmental control mechanism 350 comprises the fluid outlet 302 configured to communicate with an aperture of the food holder thereby to couple a fluid flow between the fluid outlet and an interior of the food holder. The aperture suitably communicates with the first region. Thus, the storage unit is adapted to control the environment within the first region by controlling the fluid flow (such as airflow, for example of chilled air) directed towards and/or into the first region.

In some examples, the environmental control mechanism comprises a fluid inlet 310 configured to communicate with an interior of the food holder thereby to provide a return fluid flow path for fluid flow from the fluid outlet 302.

The fluid outlet 302 of the environmental control mechanism is configured to communicate with a first aperture 216 of the food holder 130 and the fluid inlet 310 of the environmental control mechanism is configured to communicate with a second aperture 218 of the food holder, thereby to couple a fluid flow between the fluid outlet and the fluid inlet through the food holder. Suitably the fluid flow is along the fluid flow path through the first region of the food holder between the first aperture and the second aperture of the food holder.

In some examples, one or both of the fluid outlet and the fluid inlet are configured for sealing engagement with respective portions of the food holder. For example, the fluid outlet can sealingly engage with the food holder about the first aperture. The fluid inlet can sealingly engage with the food holder about the second aperture. Such sealing engagement can restrict fluid flow to being directed along the fluid flow path through the first region. This can provide for more efficient control of the environment within the first region since a greater relative proportion of the fluid flow will be along the fluid flow path compared to examples in which there is no, or a reduced, sealing engagement between one or both of the fluid inlet and fluid outlet with the first region of the food holder.

In some examples, sealing engagement between the fluid inlet and/or fluid outlet and the first region of the food holder can be effected by the provision of a resilient element on the fluid inlet and/or fluid outlet. In some examples, a resilient sealing element can be provided on or as part of the food holder. For example, a resilient O-ring can be provided around one or more aperture of the food holder. A resilient O-ring can be provided around one or both of the fluid inlet and fluid outlet, and configured to engage with a food holder received into the storage unit.

Suitably the storage unit is configured to control one or more of temperature, humidity and flow rate of fluid emitted from the fluid outlet. Thus, the storage unit can control one or more of the temperature and humidity of the first region of the food holder.

In some examples, the storage unit comprises a reader 197 configured to read an identifier 198 associated with the food holder, the storage unit being configured to control the environment within the first region in dependence on the identifier. As schematically illustrated in FIG. 1, the food holder 130 can comprise the identifier 198.

The identifier can be a food holder identifier which can identify the food holder. Such a food holder identifier can uniquely identify a food holder. The storage unit suitably comprises a communication module 150 configured to access a memory, such as a remote memory 140, for example one accessible in or through the cloud 170. The memory may comprise data (for example at a database 180) relating to that food holder, for example the foodstuffs that are stored on that food holder. The location of the foodstuffs, for example their location in the food holder, such as which region the foodstuffs are stored in can also be stored in the database. This permits the identifier to be able to identify, for each food holder, which foodstuff (or which food packaging) is stored in that food holder, and the location in the food holder.

For example, at a food packaging plant, or elsewhere, a determination can be made of the foodstuffs that are placed on a particular food holder, and the locations of those foodstuffs. Data relating to those foodstuffs and their locations can be stored in the memory so as to be associated with that particular food holder. One convenient way of achieving this is to store that data together with the food holder identifier.

The data relating to the foodstuffs can comprise storage requirements of one or more of the foodstuffs placed on the food holder. The storage requirements can comprise a maximum temperature at which the foodstuff should be stored. For example, the data may indicate that a vegetable portion should be stored at or below 8 degrees Celsius. In another example, the data may indicate that a portion of dry rice should be stored at room temperature.

In some examples, the identifier might indicate the environmental storage requirements for regions of the food holder. For example, the identifier could indicate that the first region of the food holder should be maintained at 8 degrees Celsius. The identifier may further indicate that the second region of the food holder should be allowed to remain at room temperature. This approach avoids the need for the storage unit to access the memory which, as mentioned, might be located remote from the storage unit. Arranging for the identifier to indicate the environmental storage requirements can improve the robustness of the system, for example in situations in which access to a remote memory may be interrupted.

In other examples, the identifier can comprise one or more food packaging identifier. For example, where first food packaging is located in the first region of the food holder and second food packaging is located in the second region of the food holder, the identifier can comprise a first food packaging identifier, for identifying the contents of the first food packaging and a second food packaging identifier for identifying the contents of the second food packaging. The respective food packaging can comprise the respective food packaging identifiers.

Similarly to the discussion in respect of the food holder identifier, the food packaging identifier may comprise an identifier permitting a look up of data relating to the contents of that food packaging in a memory, which might be a remote memory. Suitably data relating to the contents of the food packaging are associated in the memory with that food packaging, for example by being associated with a food packaging ID which identifies that food packaging. Alternatively or additionally, the food packaging identifier may indicate the environmental storage requirements for the food packaging. For example, the food packaging may indicate that it should be stored at or below 8 degrees Celsius.

The environmental storage requirement can comprise the temperature at which one or more of a region of the food holder, food packaging and a particular foodstuff should be stored. The requirement may indicate a maximum storage temperature. The environmental storage requirement can comprise the humidity at which one or more of a region of the food holder, food packaging and a particular foodstuff should be stored. The requirement may indicate a maximum and/or a minimum storage humidity. The environmental storage requirement can comprise the desired flow rate of a fluid through or across one or more of a region of the food holder, food packaging and a particular foodstuff.

The environmental control apparatus can, in dependence on the stored foodstuff (for example one or more of the shape, size, volume, weight, and so on, of the foodstuff), control the environment within the region of the food holder at which that foodstuff is stored.

The storage unit may, in some examples, be further configured to control the environment within the second region in dependence on the identifier.

The identifier can comprise any convenient form of identifier, such as an RFID identifier, an NFC identifier, and an optically-readable identifier (such as an identifier recognisable by image recognition systems), or any combination of these identifiers. An example of an optically-readable identifier is a barcode, such as a 1D or 2D barcode. Providing an identifier that is readable via one or more of RFID, NFC and optically permits the reading of the identifier from a location spaced from the identifier itself. The reading of the identifier can be restricted to a location near to the identifier. For example, where an NFC identifier is provided, the reader will need to be close to the identifier to be able to read it. This can reduce the chances of the reader inadvertently reading a different identifier from the desired identifier, and so increase the accuracy of the environmental control based upon such reading of the identifier.

The storage unit is suitably configured to generate an indicator signal thereby to cause an indicator to display an indication of the controlled environment. The indicator signal, in some examples, comprises an indication of the environmental condition of each of the first and second regions of the food holder. The indicator signal can cause the indicator display of the food holder and/or the further indicator display of a mobile device to display the indication.

The storage unit comprises a fluid cooler for controlling the temperature of the fluid flow, for example airflow. The storage unit can, in some examples comprise a fluid heater for controlling the temperature of the fluid flow, for example airflow. The provision of a fluid cooler and/or a fluid heater permits the temperature of the fluid flow to be maintained at a desired temperature, or at a desired temperature profile that need not be a constant temperature, irrespective of the ambient temperature of the surroundings. For example, a food holder can initially be located in a packaging plant at 20 degrees Celsius, then transported in a lorry at a temperature of 10 degrees Celsius, before being stored in a user location with an ambient temperature of 18 degrees Celsius. Where the desired temperature for a foodstuff in that food holder is 8 degrees Celsius, it will be necessary to cool ambient air in each stage to 8 degrees Celsius before feeding this cooled air to the flow path directed towards the food holder region containing that foodstuff. The amount of cooling of the ambient air will, however, differ at each stage, as will be appreciated. In another example, the transport may occur during winter, where the ambient air temperature may be below the desired temperature—the ambient air may be 2 degrees Celsius for example. In this case, it will be necessary to heat the ambient air to 8 degrees Celsius, before feeding this air to the flow path directed towards the food holder.

In some examples, the desired temperature profile can be expressed in terms of a maximum temperature. A minimum temperature need not be specified. In such examples there is no need to provide a fluid heater, as the provision of a fluid cooler will be sufficient to maintain the temperature at or below the specified maximum temperature.

The storage unit can comprise a humidifier for controlling the humidity of the fluid flow, for example airflow. The storage unit can comprise a dehumidifier for controlling the humidity of the fluid flow, for example airflow. Similarly to the situation described above in respect of temperature, to maintain the humidity of the fluid within a desired maximum and/or minimum level, it may only be necessary to provide one of the humidifier and the dehumidifier, depending on the humidity of the ambient air.

The storage unit can comprise a fluid flow control device such as a fan with an adjustable speed for controlling the flow rate of the fluid.

The control of the temperature, humidity and flow rate can be achieved so as to more quickly obtain the desired environmental condition. For example, where a food holder is in a location with an ambient air temperature of 20 degrees Celsius, and it is desired to maintain the temperature of the airflow to the container at between 4 and 8 degrees Celsius, the temperature of the airflow can be reduced to below the desired temperature initially, and an increased fan speed used, so as to accelerate to reduction in temperature. When the temperature of the food holder or foodstuff in that food holder approaches the desired temperature, the fan speed can be reduced and the airflow temperature maintained at, or slightly below, the desired temperature. An ability to quickly react to changes in the ambient air temperature, and/or humidity, can permit the environmental condition within the container to be maintained more consistently.

In some examples a filter can be provided at one or more of an air intake into the storage unit, the storage unit outlet and the food holder aperture. The provision of such a filter can enhance the cleanliness of air passed to the food holder. This can reduce the risk of contaminants contaminating food contained within the food holder.

### Nutritional Content Determination

A rough idea of the nutritional content of a food item can be obtained from a standard barcode. For instance, packaging of a lettuce or of a fish will comprise a barcode indicating that the contents of the package are a lettuce or fish, respectively. It is possible to look up the type of lettuce and type of fish and to get a general idea of the nutritional content of that food item. This can be sufficient for some purposes, but where a user's diet is to be strictly controlled, for example where the user has a dietary condition, the general idea of the nutritional content of the food item is unlikely to be accurate enough.

Accurate determination of the nutritional content of food is important for accurately calculating nutritional intake. Accurately knowing nutritional intake can be useful in many scenarios. For example an athlete may require highly accurate nutritional intake information to best maintain or adjust their diet for peak fitness. More importantly, accurate nutritional intake information can be of critical importance to a user suffering from a health condition such as diabetes. In this example, the accurate nutritional intake information can permit the user to better moderate their intake and regulate their blood sugar using more carefully controlled volumes of insulin. This can lead to an overall benefit for such users because there may as a result be a lower fluctuation of blood sugar levels. The accurate nutritional information provided by the present techniques can feed into systems used by users such as athletes and diabetic users.

Nutritional data relating to a particular foodstuff can include the amount of carbohydrate in that foodstuff, and/or the type of carbohydrate in that foodstuff. The nutritional data can comprise the amount of protein in that foodstuff. The nutritional data can comprise a glycaemic index of that foodstuff.

One approach to determining the nutritional content of food packaging (i.e. food contents of that food packaging) is to base the determination both on the type of food and a parameter relating to the particular food in the food packaging. This approach enables a more accurate determination to be made of the nutritional content of the food packaging.

In one example, a system 600 is provided for determining the nutritional content of food packaging. The system comprises a communication module 602 configured to receive an identifier associated with food packaging 604. The system also comprises a processor 606 configured to access, in dependence on the received identifier, data relating to the food packaging associated with the identifier. The data is suitably stored at a memory 608. The data comprises an indication of the type of food 610 stored in the food packaging. For example ‘lettuce’ or ‘trout fillet’. The data also includes a parameter 612 relating to the food stored in the food packaging. The parameter is suitably one determined at the time of packaging the food in the food packaging. The system is configured to determine, in dependence on the accessed parameter, the nutritional content of the food packaging identified by the identifier.

The parameter suitably comprises one or more of a weight of the food in the food packaging, a volume of the food in the food packaging, an indication of the atmosphere in which the food in the food packaging was packaged, and a date on which the food in the food packaging was packaged. In some examples the parameter comprises one or more of an amount of carbohydrate, a type of carbohydrate, an amount of protein and a glycaemic index.

Knowledge of the weight and/or volume of the food in the food packaging on packaging of that food item can further enhance the accuracy and/or reliability of the nutritional information determined. This is especially so for food items that are typically counted by ‘unit’, such as lettuce, cucumber, tomatoes and fish, amongst others. It is usual to provide an amount of each of these food items that is not necessarily linked to its weight. For example, a lettuce can be sold as an individual item (e.g. one iceberg lettuce) rather than as an amount of lettuce defined by its weight. Similarly, it is common to buy a whole number of pre-packaged cucumbers or tomatoes rather than an amount of these items defined by their weight. Fish is also a foodstuff that is typically sold as an item. A rough indication of the weight of the fish might be provided, but this is not necessarily accurate. Rather, a fish portion might be defined by the number of items—for example two trout fillets. Such a fillet would typically be of a fairly standard size. However, knowledge of the weight of such food items enables the nutritional content of those food items to be more accurately determined.

Thus, in an example of the present techniques, the precise weight of a portion of tomatoes, or the precise weight of a fish fillet is known. This can be measured at the point in the production of the food packaging at which the food item is readied for packing into the food packaging or when the food item has been put into the food packaging.

This weight is suitably stored by the system in a memory. Each food packaging will be associated with a unique food packaging identifier. This can be a simple alphanumeric identifier, or indeed any other suitable identifier. In the example above relating to a portion of fish, the food packaging may have identifier FP0001. On packaging the fish portion into this food packaging, the system can update the memory to associate food packaging identifier FP0001 with the food type ‘fish’ (this is likely to be more specific that just generally ‘fish’, for example giving the type of fish and the part of the fish comprising the fish portion; the food type ‘fish’ is used here for illustrative purposes) and the weight of that fish portion on packaging. Thus, where the weight of a fish portion might vary by ±5% around an average weight, there could potentially be a 10% difference in weight between two similar fish portions. This weight variability would lead to a corresponding nutritional content variability. Determining the nutritional content in dependence on the measured weight of the food item (here, a fish portion) can therefore greatly reduce the variability in the determination of the nutritional content.

Similarly to the discussion of the weight of a food item above, the packaged volume of a food item can be used to accurately determine the nutritional content of that food item.

The atmosphere in which a food item was packaged can affect its nutritional value. For example, where the food item is packaged in air, the presence of oxygen can lead to the nutritional content of the food item changing over time. Where a food item is packed in a different atmosphere, the change of the nutritional content over time is likely to be different. Thus storing in the memory an indication of the atmosphere in which the food item was packaged can lead to an increase in the accuracy of determining its nutritional content.

The nutritional value of a foodstuff can vary with the amount and/or type of carbohydrate present.

Thus knowledge of such information relating to the carbohydrate content permits an increase in accuracy of the nutritional content determination. Similarly, the amount or proportion of protein present can affect the nutritional content, and knowledge of such information relating to the protein content permits a more accurate determination of the nutritional content. Knowledge of the glycaemic index of a foodstuff can also permit the nutritional content determination to be more accurate.

As mentioned above, the nutritional content of food in food packaging may vary over time. Thus, simply indicating the type of food item (as in a conventional barcode) may only permit a rough idea of the nutritional content of that food item, since the ‘age’ of that food item, i.e. the length of time for which it has been packaged, would not then be available. In contrast, in the present techniques, the date on which the food is packaged into the food packaging, and optionally also the time of day, can be stored. This information is suitably stored in the memory so as to be associated with the food packaging identifier (e.g. FP0001) associated with that food packaging.

Thus, the present techniques permit knowledge of one or more of the date (and optionally time) on which a food item was packaged, the atmosphere in which the food item was packaged, a weight of the food item as packaged and a volume of the food item as packaged. Additionally or alternatively, the present techniques permit knowledge of one or more of an amount of carbohydrate, a type of carbohydrate, an amount of protein and a glycaemic index. Knowledge of one of these parameters enables a more accurate determination to be made of the nutritional content of the food packaging. Knowledge of a combination of more than one of these parameters can further enhance the accuracy with which the determination of the nutritional content can be made.

The communication module 602 is suitably configured to receive an indication of the temperature profile of the food packaging over time and the processor 606 is suitably configured to determine the nutritional content of the food packaging in dependence on the received temperature profile.

In some examples, the food packaging 604 can comprise, or be associated with, a sensor 614 configured to sense the temperature of the food packaging. The sensor may sense the temperature of the food packaging itself, for example a wall of the food packaging. The sensor may sense the temperature of the surroundings of the food packaging, for example a food holder in which the food packaging is stored.

The temperature profile may indicate the temperature at which the food packaging is stored between packaging and use. The temperature profile may indicate the temperature at which the food in the food packaging is prepared before consumption. For example, where food packaging is configured to be heated thereby to cook the food contained in that food packaging, the sensor can sense the temperature profile during cooking. This can usefully be in addition to the temperature during transport and/or storage before cooking. This approach is useful in examples where the food packaging is such that the food contents of the food packaging can be cooked in that food packaging. For example, the food packaging can be at least partly oven-proof or microwave-proof.

The nutritional content of a food item can depend strongly on the temperature to which that food item is exposed. For example, where a food item is refrigerated during storage, the nutritional content of that food item can remain at an initial level, or it might change slowly relative to the initial level. Where a food item is stored at ambient temperature, the nutritional content of that food item can change more quickly, for example as the food item begins to go off.

Further, during cooking, where food is typically raised to an elevated temperature for a prolonged period of time, the nutritional content of that food can change even more quickly. In an illustrative example, a portion of vegetables might be consumed raw, lightly cooked before consumption, or cooked thoroughly before consumption. In each case the nutritional content of the vegetable portion as consumed will differ. Thus, rather than use a typical nutritional content of a given food item as a guide, the present techniques permit a more accurate determination of the nutritional content of the food as consumed.

As mentioned elsewhere herein, such an approach can be important for regulating a carefully controlled diet, such as a diet used to manage a dietary condition. The present approach is therefore of relevance to those on a strict diet, for example those attempting to lose weight or to get healthier, athletes on a training regime and diabetic users.

In some examples, the system is configured to output a signal indicating the nutritional content of the food packaging. The system may provide an indication of the nutritional content of the food in the food packaging, for example on a display on or associated with the system.

The signal can, in some examples, indicate one or more of a carbohydrate content of the food packaging, a glycaemic index of the food in the food packaging, a weight of the food in the food packaging and so on. In general, the signal can indicate one or more aspect of the food packaging, the contents of the food packaging and nutritional data associated with the food.

In some examples the system can output a signal indicating the nutritional content for use by other devices or systems. For example, the signal may be received by a mobile device such as a mobile telephone, a tablet computer or a laptop computer. The signal may be received by an app running at a mobile device. The signal may be received by a health app, for example an app accessible via Amazon's Alexa interface or Apple's Sin interface. The signal may be received by a device configured to automatically administer insulin to a user. Additionally or alternatively, an app or other control system, configured to receive the signal, may be arranged to effect control over a device for automatically administering insulin. One or more of the system, the mobile device, the app or other control system and the device may use the nutritional content indicated by the signal to make calculations relating to other food that the user might consume at the same time, and/or to make calculations relating to actions that the user might take in response to consuming the food and/or automatic actions to be taken in response to the user consuming the food. One example of this, in the context of a diabetic user, is for the calculation to relate to a volume of insulin to be injected, and/or a time at which insulin should be injected. The result of this calculation may be provided to a user for manual administration of insulin. The result may be provided to the automatic insulin administration device so as to cause that device to automatically administer an appropriate volume of insulin at an appropriate time. Suitably the automatic insulin administration is subject to confirmation by a user. For example, the system or app may automatically determine the volume and timing of delivery of insulin and present a user with a request to authorise the delivery. For example, before each separate delivery of insulin into a user's body, the system or app may require authorisation from a user to proceed with that delivery.

The communication module is suitably configured to receive a plurality of identifiers, each identifier being associated with respective food packaging. The processor is suitably configured, in dependence on the respective identifier, to access data relating to the respective food packaging and determine the nutritional content of each of the plurality of food packaging. In some examples, the system is configured to output a signal indicating the nutritional content of the plurality of food packaging.

This approach permits a selection, or subset, of the available food in different food packaging to be made. For example a selection can be made between different food packaging that a user has in their kitchen. The available food packaging can vary over time as the user consumes some food items, and buys new food items. The present approach permits the selection to be based on a currently available set of food packaging. A determination can be made of the nutritional content of each item of food in the available food packaging separately, and a combination of the food items in the available food packaging. This allows a user, or an app interfacing with the system, to easily ascertain the nutritional content of a meal comprising food contained in the food packaging.

In some examples, the system comprises a reader 616 for reading the identifier or the plurality of identifiers. The reader is suitably configured to send the identifier or the plurality of identifiers to the communication module 602. The reader can be remote from the memory 608 and/or from the communication module 602.

In some examples, the identifiers read by the reader can indicate the available food packaging.

In some examples, the reader 616 can be provided at a food holder and/or at a storage unit. The reader may, in some examples, be provided on a mobile device, such as a mobile telephone, a tablet computer or a laptop computer. The reader can be used to read the identifier on use of the food packaging, for example when preparing a meal using the food packaging. Where the reader is provided at the food holder and/or at the storage unit, the reader may be activated to read the food packaging identifier when it is determined that the food packaging is being removed from the food holder and/or from the storage unit. In one example, the food holder can detect the presence of food packaging by using a weight sensor. Where this weight sensor detects that the food packaging has been removed from the food holder, the reader may be activated to read the identifier of that food packaging. This approach permits the automatic identification of food packaging that is removed for use by a user. This approach reduces the burden upon the user to manually scan the food packaging, and so can increase the accuracy of the system by avoiding error introduced by a user forgetting to scan food packaging before consuming its contents.

The communication module may be local to the storage unit. In other examples the communication module 602 is remote from the storage unit. The communication module may be located at a server 618. The reader 616 may communicate with the communication module 602 over a network 620 such as a WAN, a LAN or the internet.

The memory 608 is, in at least some examples, provided at a server 618 remote from the reader 616. The memory can comprise a database 622 storing the data relating to the food packaging.

A method of determining the nutritional content of food packaging will now be described. The method comprises receiving an identifier associated with food packaging 702. Data relating to the food packaging 704 associated with the identifier is accessed 706. The access is made in dependence on the received identifier. The accessed data comprises an indication of the type of food stored in the food packaging. The accessed data also includes a parameter relating to the food stored in the food packaging determined on packing the food packaging. The method includes determining 708, in dependence on the accessed parameter, the nutritional content of the food packaging identified by the identifier.

The method suitably includes receiving an indication of the temperature profile 710 of the food packaging over time and determining the nutritional content of the food packaging in dependence on the received temperature profile.

A signal may be output 712, which indicates the nutritional content of the food packaging.

The method suitably includes receiving a plurality of identifiers, each identifier being associated with respective food packaging and, in dependence on the respective identifier, accessing data relating to the respective food packaging and determining the nutritional content of the plurality of food packaging. A signal can be output indicating the nutritional content of the plurality of food packaging. The method may comprise reading the identifier or the plurality of identifiers.

### Selecting Foodstuffs

In dependence on the determined nutritional content of food packaging, or otherwise, it is possible to select a foodstuff (e.g. food packaging containing food) from a plurality of foodstuffs (e.g. a plurality of food packaging containing different food). This selection can be made as part of a carefully controlled diet. For example, a particular food can be selected so as to fulfill a user's daily nutritional needs and/or to help regulate a user's physiological condition. An example of this is to help regulate a user's blood sugar level. This can be important for maintaining a user's energy throughout a day, and particularly important for those with a dietary condition such as diabetes.

The selection can be based on several factors, including food data relating to the food items. The selection can also be based on a user's physiological data and the user's activity data. This can provide information relating to how active the user is, and how the different food items for selection may affect the user, for example in terms of how they may affect that user's physiology.

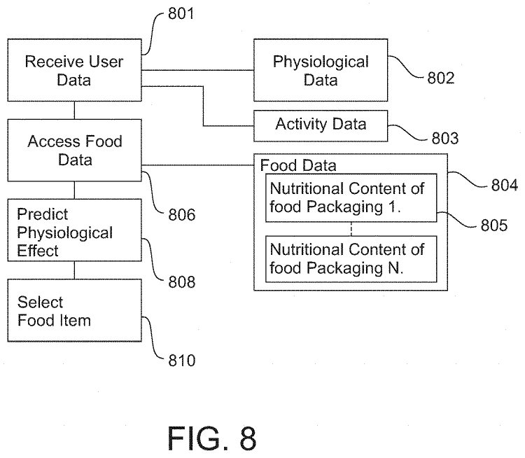

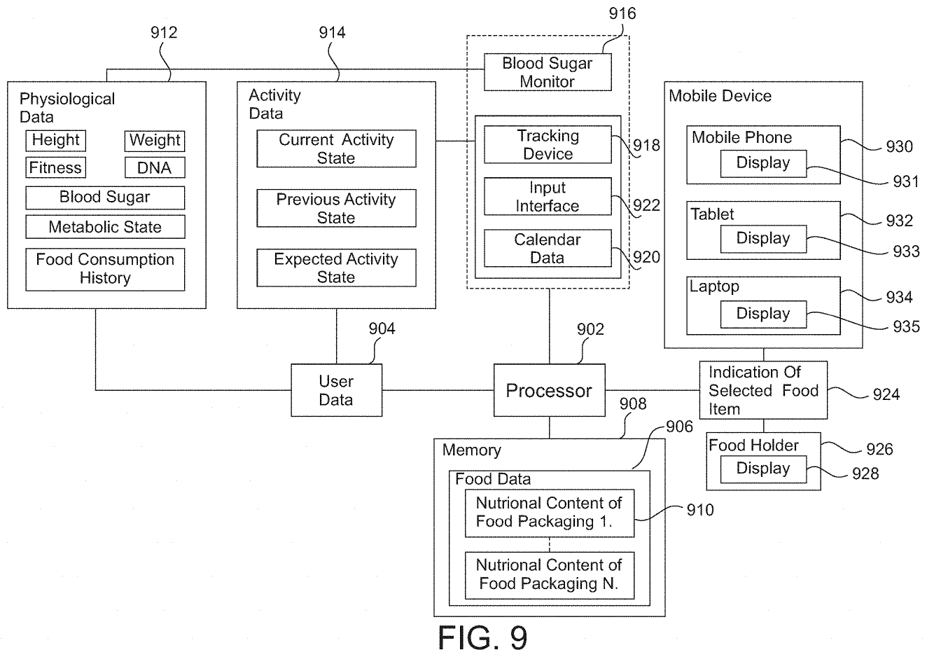

A method for recommending a food item from a group of food items for consumption by a user will be discussed with reference to FIG. 8. A system for implementing the method will be discussed with reference to FIG. 9. The food item can be contained in food packaging. For example, the method is suitably for recommending food packaging from a plurality of different food packaging, the contents of the recommended food packaging being for consumption by a user.

The method comprises receiving 801 user data 802, 803 relating to the user. Food data 804 relating to the group of food items is accessed 806. The food data 804 comprises nutritional content 805 of each of the food items in the group of food items.

The method comprises determining a predicted physiological effect on the user of each food item of the group of food items 808. The predicted physiological effect is determined in dependence on the food data 804 and the user data 802, 803. The method further comprises selecting, in dependence on the predicted physiological effect on the user, one or more food item of the group of food items for consumption by the user 810.

This approach permits a recommendation to be provided that is tailored to a particular user. Further, the recommendation can be tailored to that user's needs at any given time, for example by being based on current user data associated with the user.

FIG. 9 illustrates an example of a system for recommending a food item from a group of food items for consumption by a user. As illustrated, the system comprises a processor 902. The processor is configured to receive user data 904 relating to the user. The processor is also configured to access food data relating to the group of food items 906. The food data is stored at a memory 908 accessible to the processor. The food data comprises data relating to the nutritional content of each of the food items in the group of food items 910. The processor is configured to determine a predicted physiological effect on the user of each food item of the group of food items in dependence on the food data and the user data. The processor is further configured to select, in dependence on the predicted physiological effect on the user, one or more food item of the group of food items for consumption by the user.

In some examples, the user data can include physiological data relating to the user 802, 912. In some examples, the user data can include activity data relating to the user 803, 914. The data can include data relating to food consumed by the user within a predetermined period of time preceding the selection of the food item for consumption by the user.

Physiological data can provide information about the physical parameters or the physical status of the user. Physiological data can provide information about how the user is likely to react to a given food item. Activity data can provide information about the recent, current or future activity state of the user. This can impact on how the food to be consumed is likely to be metabolised by the user. For example, if the user has recently undertaken a vigorous activity, the food to be consumed may need to be metabolised more quickly to satisfy one or more predetermined criteria relating to the user, such as blood sugar level for example. A quick-release food item may, in such circumstances, be more appropriate than a slow-release food item. In another example, if the user is shortly expected to undertake a vigorous activity, then the food to be consumed may need to contain sufficient energy for the user but may be able to be metabolised more slowly so as to help regulate one or more predetermined criteria relating to the user, such as blood sugar level. In this latter example, a slow-release food item may be more appropriate.

In some examples, the physiological data relating to the user comprises one or more of a height and/or weight of the user, a fitness level of the user, at least a portion of a DNA profile of the user, a blood sugar level of the user, a metabolic state of the user, and food consumption history of the user. Other physiological data can be provided as appropriate, as would be understood.

The physiological data can comprise data based on current physiological data. The physiological data can comprise data based on previous physiological data, e.g. historical physiological data. The historical physiological data may be specific to that particular user. The historical physiological data may be specific to a group of which that user is a member. For example a family group. One sibling may have a similar physiological response to another sibling. One sibling's historical data may therefore be used to provide a recommendation for the other sibling.

It can be determined that the user belongs to a particular group of users based on one or more of several factors. Groups of users can include family groups (where biologically related, at least some physiological aspects are likely to be similar for different members of this group), school classes or sports groups (where the group members regularly carry out activities together, they may have similar fitness levels for example, and may have consumed the same meals), groups determined in dependence on DNA profiling (here, members of such groups are likely to share physiological traits which are based on aspects of similar DNA profiles; such traits might include metabolic rates and so on).

The method can comprise determining the blood sugar level of the user in dependence on a signal from a blood sugar monitor 916. The blood sugar monitor may be a portable device usable by the user for regularly checking their blood sugar level. The blood sugar level measured by the blood sugar monitor can be sent over one or both of a wired and a wireless (e.g. one or more of WiFi, Bluetooth, NFC) connection. The blood sugar monitor may be manually operated, e.g. the user may need to manually check their blood sugar level at various points during the day. An output signal from the blood sugar monitor can be sent, for example wirelessly, to a receiver, for example a receiver forming part of the recommendation system. The processor is suitably configured to access data from the blood sugar monitor 916. In some examples, the blood sugar monitor can be configured to periodically send data indicative of blood sugar value, for receipt by the processor. The periodic data transmission might, for example, be every 5 minutes, every 2 minutes, every minute, and so on. In some examples, the data transmission can be continuous, or nearly continuous (for example once every 10 seconds, or more frequently such as once per second), for example so as to ensure that the processor always has up-to-date blood sugar data. This approach represents a ‘push’ of data from the blood sugar monitor to the processor. Another approach is a ‘pull’ approach, where the processor requests data from the blood sugar monitor.

The processor may be configured to request data indicative of blood sugar value periodically (for example every 2 minutes or so), continuously or nearly continuously (for example at least once every 10 seconds). A combination of ‘push’ and ‘pull’ approaches can be used in some examples.

A data value may be used for the user that represents the value of a parameter at an instant in time, for example the present instant in time. For example the user's current height and weight, and/or the user's current blood sugar level. The physiological data may comprise an average of physiological data over a predetermined period of time preceding the selection of the food item for consumption by the user. Use of an average value can help smooth out spikes in the data which might otherwise lead to less accurate recommendations. A historical profile of a value can be used. This is less likely to be relevant for a user's height, but is likely to be more relevant for a user's blood sugar level. Knowledge of past values of blood sugar level are likely to inform the recommendation. Knowledge of such a profile will reveal more information than a simple average value.

In some examples, the activity data relating to the user comprises one or more of a current activity state of the user, a previous activity state of the user, and an expected activity state of the user. Use of such activity data, or combinations of such activity data, permit the user's activity state to be taken into account. For example, where the user has recently completed an activity, or is about to start an activity, the user's calorific requirements are likely to be greater. Basing a recommendation of food packaging for consumption on the user's activity state is therefore likely to enable a more stable regulation of the user's physiological data, for example their blood sugar level.

In another example, where a user is to start an activity in 1 hour's time, a relatively fast-release foodstuff might be appropriate. Where a user is to start an activity in 3 hours' time, a relatively slow-release foodstuff might be appropriate. The foodstuff to be selected may also depend on the length and/or type of the activity. For example, a user will need more energy for a 3 hour run than for a 1 hour game of table tennis.

The activity data can be determined in dependence on one or more of manually input data relating to at least one of a current activity, a previous activity and an expected activity, a tracking device 918, and a calendar entry 920. This approach enables flexibility in the way in which the method can make use of the activity data. Suitably the processor is configured to access data from one or both of the tracking device and a calendar.

The tracking device 918 may be a fitness tracking device that is able to determine an activity level and/or heart rate of the user. The calendar entry 920 may indicate that an activity has taken place or is due to take place. For instance, if there is a calendar entry on Wednesday evening at 6.30 pm till 7.30 pm for football practice, then, if the determination of the food packaging for consumption by the user is made at 5 pm on Wednesday, it can take account of the forthcoming activity. Similarly, if the determination is made at 8 pm, it can take account of the activity that has recently been completed.

In some examples, the method may comprise determining that a change in the user's physiological data has occurred which indicates that an activity has been undertaken by the user, requesting user input relating to the indicated activity, and storing one or both of the day and time of day together with the user input, and subsequently determining the activity data in dependence on the stored data.

This approach of letting the system ‘learn’ information regarding the user permits a more user-friendly interface and set-up to the system. For example, one way to set up the system would be to exhaustively input all the regular activities undertaken by a user, the day and the time of day that those activities are undertaken. This places a burden on the user to remember all the activities that they are likely to undertake, and to accurately enter all of the data. Another way to set up the system would be to provide a more limited set of data to the system, but to provide the ability for the system to detect unexpected events and to learn the user's behaviour from these events. Such learning might be inferred by the system. Such learning might be achieved by prompting the user for user input at the appropriate time, as given in the example above. The learning may be achieved by a combination of these approaches. User input can be provided by way of an input interface 922, coupled to the processor.

Whilst prompting a user for input will require the user to take action to update the activity information, this is likely to require less input than exhaustively setting the system up beforehand. For instance, where setting the system up beforehand, a user may need to input a day of the week, a start time, an activity and an end time. The user may also need to input a repetition frequency, e.g. once per week, for the activity. Where reacting to a prompt for information when the system has detected an unexpected activity (for example using a tracking device, or analysing blood sugar levels) the user may simply need to indicate the activity, because the day, start time and end time can automatically be determined by the system. The user may indicate the repetition frequency of the activity, or the system may infer the repetition frequency in dependence on the frequency at which it detects the activity.

The system can also improve its accuracy over time, by ‘learning’ data relating to the particular user. For example, on initial setup of the system, inferences may need to be made based, for example, on characteristics of one or more group of which a user is a member. An example of this is use of a DNA profile. A set of initial data can be provided to the system in dependence on a DNA profile of a user. This set of initial data may, for example, represent typical data for users with similar DNA profiles. After some time has elapsed, the system will have access to data relating specifically to that user, and so can base future determinations on that data rather than on inferred data, or the set of initial data. The system may be configured to weight the data so that as time progresses and/or as more specific user data is obtained, the proportional weighting applied to specific user data, rather than more general data, increases.

Suitably, the method comprises monitoring the user's physiological response to a particular food item, and selecting the food item for consumption by the user in dependence on the user's previous physiological response to that particular food item.

For example, where the user's blood sugar level changed by, say, +2 mmol/l after consuming a given food item, this previous change can be taken into account when determining which food item to recommend—whether that given food item or another food item. For example, if the system determines that the user's blood sugar level should ideally be increased by 2.5 mmol/l, then consuming that given food item may form at least a part of the recommendation. In another example, if the system determines that the user's blood sugar level should ideally be increased by 1.0 mmol/I, then consuming an alternative food item (or combination of food items) that is predicted to lead to an increase in blood sugar level closer to 1.0 mmol/l may form part of the recommendation.

The selection of the one or more food item of the group of food items for consumption by the user comprises determining a proportion of a food item for consumption by the user. In some situations, the food item nutritional content may not exactly match the desired nutritional intake determined by the system. In such examples it may be appropriate to suggest to the user that a portion of a food item is consumed rather than the whole food item. For example, where one food item contains potato salad, the recommendation may comprise consuming half of the potato salad. The remaining half can be replaced in the food holder for future use. Suitably the food holder can determine the weight of the food item replaced in the food holder and can thereby determine the proportion of the food contents of that food item remaining. This value can be fed back into the system for recommending food items, so that a more accurate determination can be made of the food that a user has previously consumed.

The method can comprise generating a signal indicative of the selected one or more food item 924 of the group of food items for consumption by the user, thereby to cause an indicator to indicate the selected one or more food item. The signal can indicate data relating to the selected one or more food item, such as the total carbohydrate and/or protein content of the selected item(s). Such data can be cumulative in respect of food items selected as part of a single meal, as part of meals over the course of a day, and so on, as desired. The signal can indicate the nutritional content of the food items, and/or the cumulative nutritional content of selected food items over the course of a meal, a day, and so on. Thus, the signal can cause the indicator to display the data relating to the selected food item.

The signal may be suitable for allowing the system to interact with health apps or other third party devices, directly and/or via, for example, Alexa, Sin and so on.

The signal can be output for display, for example on one or both of a local and remote display.

In some examples, the method comprises receiving the signal at a food holder 926 containing at least one food item and causing an indicator display 928 of the food holder to indicate the selected one or more food item. In some examples, a food holder can comprise a display or indicator on an exposed portion thereof. Suitably the display or indicator of the food holder containing a determined food item can be caused to indicate this fact to a user. Such indication can comprise a visual indication such as one or more of a flashing light, displaying the determined food item on the display and so on. Such an indication can comprise an audible indication such as a beeping noise for drawing the user's attention to that food holder.

The method may comprise receiving the signal at a mobile device 930, 932, 934 and causing an indicator such as a display 931, 933, 935 at the mobile device to indicate the selected food item and/or data relating to the selected food item. The mobile device can comprise one or more of a mobile telephone 930, a tablet computer 932 and a laptop computer 934. The mobile device may be running an app which can receive the signal. The display of the mobile device can display the indication of the determined food packaging.

### Food Packaging

Knowledge of when a user consumes food contained in food packaging can greatly increase the accuracy of systems for determining a user's food intake and actions based thereon, for example administering insulin.

Conventional systems can require a user to scan a barcode on food packaging. The system will then assume that the contents of that food packaging are shortly to be consumed by a user. However, the user may change their mind and not consume that food after all. The food packaging may be replaced in a storage location without it being re-scanned. Thus the system will not have accurate knowledge of the food consumed. Alternatively, the user may take the food item and carry it with them for later consumption. Again, the system may erroneously consider the food to have been consumed at an earlier point in time than that at which it is actually consumed.

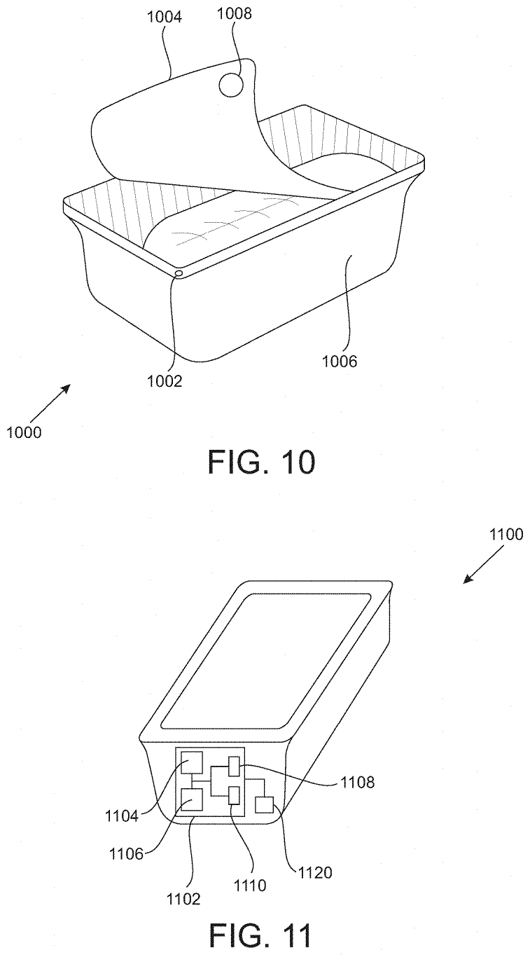

It is therefore desirable for a system to more accurately be able to determine consumption of a food item contained in food packaging. This can be achieved by the provision of food packaging that is able to indicate a state of readiness of the food packaging for consumption of the food contained in the food packaging. An example of such food packaging is illustrated in FIG. 10 at 1000. Suitably a tag is provided on or as part of the food packaging, which tag is configured to transmit a signal indicative of the preparation of the food packaging for consumption of food contained in the food packaging. The tag is suitably configured to detect an indication of the preparation of the food packaging. In the discussion below, examples of tags will be described with reference to FIGS. 10 and 11. It will be appreciated that in some examples the features and functionality of the tags (separately described below) can be combined. Thus, a food holder is contemplated which comprises a tag with such a combination of features. They are described below separately for clarity.

In some examples, the tag comprises a sensor 1002 configured to detect the separation of one portion of the food packaging from another portion of the food packaging. For example, the sensor can detect the separation of a cover 1004 of the food packaging from a body 1006 of the food packaging. The sensor is configured to generate a separation signal in dependence on the detected separation. The signal indicative of the preparation can comprise the separation signal generated by the sensor.

The tag is suitably configured to transmit the signal indicative of the preparation in dependence on the detection of the separation. The tag may be configured such that the detection of the separation of portions of the food packaging from one another causes the tag to transmit the signal. This approach can avoid the need for the tag to continuously or periodically transmit a status signal indicating the status of separation (e.g. whether or not the sensor has detected that separation of portions of the food packaging has occurred). Rather, the tag need only transmit the signal when it is detected that separation has occurred. This can prolong the battery life of a battery provided at or connected to the tag.

Suitably, the tag comprises a proximity sensor configured to detect the separation and to generate the separation signal in dependence on the detected separation. The proximity sensor can be configured to detect the proximity of one portion of the packaging to another portion of the packaging, so as to enable detection of the separation of those portions. For example the sensor may comprise a light sensitive element. Such a sensor can be provided on a body 1006 of the food packaging. A cover 1004 of the food packaging can comprise an opaque element arranged, when the cover covers the food contained in the food packaging, to cover the light sensitive element. On opening of the cover, or at least partial opening of the cover, the opaque elements can be spaced from the light sensitive element so that the light sensitive element generates a signal indicating a change in the light condition. Such a signal can therefore indicate that the cover has been opened or removed from the food packaging.

In some examples, the proximity sensor can comprise a magnetic sensor, for sensing proximity to a magnetic material. In some examples, the proximity sensor can comprise a capacitive sensor, for capacitively sensing proximity to an electrically conductive material.

Combinations of the above-mentioned types of sensor may be used together.

Where the sensor comprises a plurality of interacting portions (for example, a light sensitive element and an opaque element, a magnetic sensor and a magnetic element, and a capacitive sensor and a capacitive element), the sensor can comprise a plurality of sensor portions. For example, referring again to FIG. 10, the sensor can comprise a first sensor portion 1002 and a second sensor portion 1008. The first sensor portion can, in some examples, be provided on the body 1006 of the food packaging 1000. The second sensor portion can, in some examples, be provided on the cover 1004 of the food packaging. The sensor is suitably configured to detect the separation of the first sensor portion from the second sensor portion, thereby to detect separation of one portion of the food packaging from another portion of the food packaging.

In some examples, the tag is configured to detect the separation of portions of the food packaging from one another in dependence on determining a change in an electrically conductive path through at least a portion of the food packaging. The tag can comprise an electrical circuit with two spaced contacts. The part of the tag comprising the circuit and the contacts can be provided on one part of the food packaging, for example on the body 1006. An electrically conductive element, for example a wire or foil sheet, can be provided on another part of the food packaging arranged for separation from the one part of the food packaging on opening of the food packaging. For example, the electrically conductive element can be provided on the cover 1004. When the cover is closed, the electrically conductive element is arranged to contact both of the two contacts thereby closing the electrical circuit between the two contacts. When the cover is at least partially opened, the electrically conductive element is arranged to separate from at least one of the contacts thereby opening that part of the circuit. This opening, or breaking, of the circuit can be detected by the tag thereby enabling detection of the separation of the two portions of the food packaging.

Some embodiments of the food packaging are microwave-proof containers. In these embodiments the food packaging can be placed directly into the microwave to enable microwave heating of the food contained in the food packaging. The tag may not be microwave-proof. For example it is likely that the tag will comprise metallic components and so will not be suitable for microwaving. In these examples, the tag can be removed from the food packaging prior to microwaving the food packaging. The removal of the tag from the packaging, i.e. the separation of the tag from the food packaging, can be detected in a manner similar to the above. For example by one or more of light, proximity, magnetic and electrical path detection. Suitably, the tag can be configured to transmit the signal indicating preparation of the food packaging on detecting that it (i.e. the tag) is removed from the food packaging.

In some examples, the tag is configured to detect a temperature of the food packaging and to generate a readiness signal in dependence on the detected temperature. The signal indicative of the preparation of the food packaging for consumption of food contained in the food packaging suitably comprises the readiness signal. The tag is suitably configured to transmit the signal indicative of the preparation in dependence on the readiness signal.

FIG. 11 illustrates an example of food packaging 1100 in which a tag 1102 is able to detect a temperature of the food packaging. The tag 1102 suitably comprises a temperature sensor 1104. The tag suitably further comprises a memory 1106 for storing temperature data, for example a temperature profile indicative of the temperature over time experienced by the food packaging.

The tag is suitably configured to generate the readiness signal in dependence on the detected temperature satisfying a temperature criterion. The temperature criterion can be stored in the memory 1106. The tag can comprise a processor 1108 configured to determine when the temperature satisfies the temperature criterion. In dependence on such a determination, the processor can control a transmitter 1110 to transmit the signal.

In some examples, the tag is configured to determine that the temperature satisfies the temperature criterion in dependence on the temperature profile of the food packaging. The processor 1108 is suitably configured to make this determination in dependence on a temperature profile stored in the memory 1106.

The temperature criterion can comprise a threshold temperature. The tag is configured to determine that the temperature criterion is satisfied in dependence on the temperature exceeding the threshold temperature. Suitably, the processor is configured to determine that the temperature exceeds the threshold temperature.

The temperature criterion can comprise a threshold thermal budget, and the tag is configured to determine that the temperature criterion is satisfied in dependence on the temperature profile indicating that the thermal budget of the food packaging exceeds the threshold thermal budget. Suitably, the processor is configured to determine that the detected thermal budget of the food packaging exceeds the threshold thermal budget.

In some examples, the temperature criterion comprises a further threshold temperature. The tag is, in these examples, configured to determine that the temperature criterion is satisfied in dependence on one or both of the temperature exceeding the threshold temperature, and the thermal budget of the food packaging exceeding the threshold thermal budget, and in dependence on the temperature subsequently falling below the further threshold temperature.

The processor can be configured to monitor the temperature of the food packaging, and to determine whether
(i) the temperature exceeds the threshold temperature, and/or
(ii) the thermal budget of the food packaging exceeds the threshold thermal budget.

The processor is, in these examples, further configured to determine whether
(iii) the temperature falls below the further threshold temperature.

An enhanced accuracy of determining use of the food packaging can be provided by monitoring movement of the food packaging. A determination that the food packaging is ready for use by a user can be made in dependence on determining movement of the food packaging. In some examples, the tag is configured to determine movement of the food packaging and to transmit the signal indicative of the preparation in dependence on the determined movement.

The tag can be configured to determine movement of the food packaging in dependence on a weight signal generated by a weight sensor. The food packaging 1100 can comprise the weight sensor 1120. The weight sensor is suitably provided at or towards a lower part of the food packaging thereby to detect when the food packaging is placed on a surface. The tag can, in some examples, comprise the weight sensor. In other examples, the tag can be coupled to a weight sensor provided on or as part of the food packaging but spaced from the tag (as illustrated in FIG. 11).

Alternatively or additionally the tag can have access, for example by a wireless communication path, such as over radio frequencies, to a remote weight sensor. The tag can receive the weight signal over such a communication path.

Thus, the weight sensor can be provided in one or both of the food packaging itself and the food holder on which the food packaging is placed. Providing the weight sensor in the food packaging itself can avoid communication issues between the food packaging and the food holder. This approach may also permit the weight sensor to provide useful information where the food packaging is not initially placed in the food holder. Providing the weight sensor in the food holder (or, more generally, at a location at which the food packaging can be placed) can reduce the complexity of the food packaging.

In some examples, the tag is configured to determine a distance from a location external to the food packaging. The tag is configured to determine movement of the food packaging in dependence on the determined distance. The tag can be configured to determine the distance in dependence on a received signal strength of a signal generated at the external location. The tag is suitably configured to receive the signal generated at the external location. The tag can comprise a receiver for receiving that signal. In some embodiments, the tag can comprise a transceiver and/or a transmitter and receiver. This arrangement can enable the tag to transmit the signal indicating preparation of the food packaging, and also to receive signals including weight signals and signals for indicating distance from an external location.

The signal indicating distance may be generated at a food holder and/or at a storage unit. The tag may therefore be configured to determine a distance relative to the food holder and/or the storage unit, respectively. This can indicate when the food packaging is removed from the food holder and/or the storage unit. The removal of the food packaging from the food holder and/or the storage unit, together with the identification by the tag of the preparation of the food packaging, can provide an enhanced indication, for example to a user or to a system for determining insulin administration, that the food contained in the food packaging is ready to be consumed by a user.

The signal indicating distance may be generated at a smart appliance, such as a smart microwave and/or a smart oven. The tag may therefore be configured to determine a distance relative to the smart appliance. This can indicate when the food packaging is placed in an oven for heating, for example. The location of food packaging in the oven, together with the identification by the tag of the preparation of the food packaging, can provide an enhanced indication, for example to a user or to a system for determining insulin administration, that the food contained in the food packaging is ready to be consumed by a user.

In some examples, the signal indicative of the preparation of the food packaging identifies a smart appliance to be used to prepare the food, for instance a smart microwave or a smart oven. An indication of the smart appliance can be conveyed to a user, such as by a display of at least one of the food holder, storage unit, mobile device and so on. The smart appliance may be configured to detect the signal and to configure the heating of the food packaging in dependence thereon.

The signal generated at the external location can comprise one or more of an RFID signal and an

NFC signal. The signal can be transmitted by an identifier external to the food packaging, for example an identifier at the food holder and/or at the storage unit. The distance may be determined in dependence on the signal strength of the RFID signal and/or the NFC signal. In examples, such as where food packaging may be removed from a food holder/storage unit, the distance can be determined in dependence on the signal strength falling below a threshold proportion of a respective steady-state signal strength. The distance can be determined in dependence on the signal strength falling below a predetermined signal strength. In examples, such as where food packaging may be placed in a smart appliance such as an oven, the distance can be determined in dependence on the signal strength increasing beyond a threshold proportion of a respective steady-state signal strength. The distance can be determined in dependence on the signal strength increasing beyond a predetermined signal strength.

Suitably, the signal indicative of the preparation of the food packaging identifies the food contained in the food packaging.

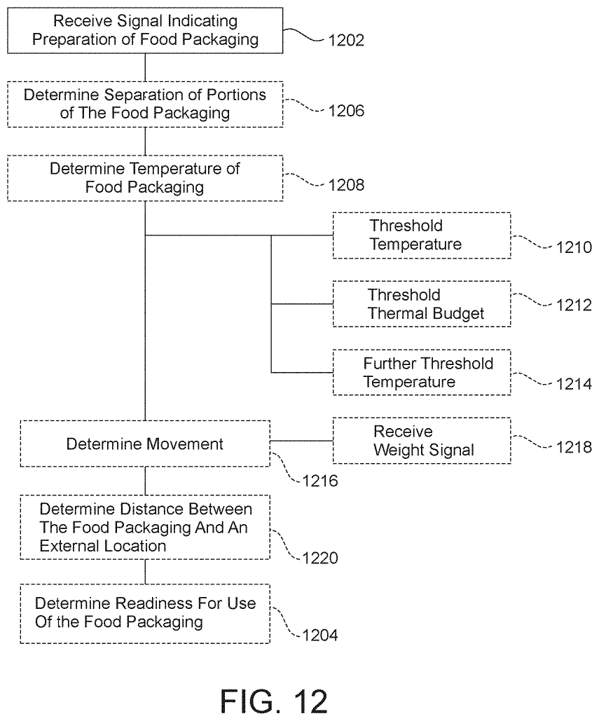

Referring now to FIG. 12, a method is provided for detecting the readiness for use of food packaging. The method comprises receiving a signal transmitted by a tag associated with the food packaging 1202. The signal indicates preparation of the food packaging for consumption of food contained in the food packaging. The method further comprises determining readiness for use of the food packaging in dependence on the received signal 1204.

The method can, in some examples, comprise determining, in dependence on the received signal, a separation between one portion of the food packaging and another portion of the food packaging 1206, and determining the readiness for use in dependence on the determined separation.

Optionally, the method comprises determining, in dependence on the received signal, a temperature of the food packaging 1208, and determining the readiness for use in dependence on the determined temperature. The method may comprise determining that the temperature exceeds a threshold temperature 1210 and determining the readiness for use in dependence thereon. The method may comprise determining a thermal budget of the food packaging based on the determined temperature and determining that the thermal budget exceeds a threshold thermal budget 1212 and determining the readiness for use in dependence thereon.

The method can also comprise determining that the temperature subsequently falls below a further threshold temperature 1214 and determining the readiness for use in dependence thereon.

In some examples, the method comprises determining movement of the food packaging 1216 and determining readiness for use in dependence on the determined movement. Optionally, the method comprises receiving a weight signal 1218, for example a weight signal generated by a weight sensor, and determining movement in dependence thereon.

The method can comprise determining a distance between the food packaging and a location external to the food packaging 1220, and determining movement in dependence thereon.

These further options have been illustrated in dashed lines in FIG. 12 to indicate that one or more of the options may be provided in the method described herein.

The system may further comprise, or couple to, an automatic insulin administration device and the method may comprise controlling such an automatic insulin administration device. On detecting that food contents of a food package are ready to be consumed by a user, the system and method can comprise determining that those food contents will be consumed by the user. In response to this determination, the system and method can determine the nutritional content of the food, for example using the techniques described herein. Knowledge of the nutritional content of the food enables calculation of how that food will, or is likely to, affect the user's physiology including their blood sugar level. In dependence on this calculation, a determination can be made of one or more volumes of insulin for administering to a user. The determined volume(s) of insulin can be determined as appropriate to help regulate the user's blood sugar levels. It may be determined that a single volume of insulin should be administered at a specified time (for example, ‘now’, 1 hour after eating, and so on). It may be determined that multiple volumes of insulin should be administered at different specified times (for example, one volume of insulin to be administered 1 hour after eating and another volume of insulin to be administered 2 hours after eating, and so on).

The system can generate a signal thereby to control the automatic insulin administration device. In this way, the system can control the automatic administration of insulin to a user. This can provide convenience for a user because they will not need to calculate volumes or times for insulin administration, nor will they have to remember to administer the insulin at the appropriate times. Thus the provision of control of the automatic insulin administration device can reduce errors in insulin administration.

In some examples, a confirmation signal may be requested from a user before insulin is actually administered. This can increase the safety of the system, to reduce the risk that insulin is inadvertently administered without a meal then being consumed by the user. In some examples, the system may be configured to accept a ‘pause’ command from a user whereby a user can indicate a delay in the consumption of the food, for example if they answer the telephone before consuming the prepared food. This can cause an appropriate delay in the timings determined for insulin delivery.

It is also possible to provide for automatic blood sugar testing, and/or to access blood sugar test data. Blood sugar test data may be taken at regular intervals, and so the blood sugar level can be known or determined (for example extrapolated from a time series of blood sugar level values). This information can also be fed into the system for providing increased accuracy of the volume(s) and time(s) at which insulin should be delivered into a user's body.

In embodiments described above, the system can detect a time and/or duration of cooking of food contained in food packaging. This can be detected, for example, based on instantaneous temperature readings (e.g. a temperature falling below, say, 70 degrees Celsius, can indicate that cooking was performed and has finished) and a temperature profile of the food packaging. Knowledge of the cooking time and/or duration can improve the accuracy with which the nutritional content of the food to be consumed by a user is calculated. Further, knowledge of the timing of cooking can provide a more accurate indication of when the food is likely to be consumed by a user. For example, where a hot meal is prepared, the food is typically consumed shortly after preparation. Thus, where it is determined that cooking of a meal has occurred at a given time, it can further be determined that that meal is to be consumed within, say, 1 hour of that given time.

More precise knowledge of the times at which food is consumed, optionally together with more precise knowledge of the nutritional content of that food, and physiological data at that moment in time (for example current blood sugar level) therefore enables a more accurate determination of the insulin volume(s) required to help regulate a user's blood sugar level.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A food holder for carrying multiple foodstuffs to be stored at different environmental conditions, the food holder comprising:
a first region for receiving a foodstuff and
a second region for receiving another foodstuff, the second region being at least partly environmentally isolated from the first region,
the food holder being adapted to afford communication between the first region and an environment external to the food holder thereby to control an environment within the first region separately from an environment within the second region.

2. A food holder according to claim 1, in which the food holder comprises a transmissive portion configured to permit the first region to communicate with the external environment.

3. A food holder according to claim 2, in which the transmissive portion forms at least part of one or more of an upper, side and lower wall of the first region of the food holder.

4. A food holder according to claim 2 or claim 3, in which the transmissive portion is configured to transmit fluid.

5. A food holder according to any of claims 2 to 4, in which the transmissive portion comprises one or more aperture.

6. A food holder according to any of claims 2 to 5, comprising:
one transmissive portion comprising a first aperture in one of the upper, side and lower walls of the first region and
another transmissive portion comprising a second aperture in another of the upper, side and lower walls of the first region, thereby creating a fluid flow path through the first region between the first aperture and the second aperture.

7. A food holder according to claim 6, in which the first aperture is a different size to the second aperture.

8. A food holder according to any preceding claim, configured to receive a fluid flow from the external environment and to couple the fluid flow into the fluid flow path through the first region.

9. A food holder according to any of claims 2 to 8, in which the transmissive portion is configured to transmit heat more readily than a remainder of the food holder, thereby to thermally equalise the environment within the first region with that of the external environment.

10. A food holder according to any preceding claim, comprising a sensor for sensing an environmental condition within one or both of the first region and the second region.

11. A food holder according to claim 10, in which the sensor comprises a first sensor for sensing the environmental condition within the first region and a second sensor for sensing the environmental condition within the second region.

12. A food holder according to claim 10 or claim 11, in which the sensor comprises a weight sensor for sensing the weight of foodstuffs within one or both of the first region and the second region.

13. A food holder according to any of claims 10 to 12, comprising an indicator display configured to receive a signal from the sensor and in dependence on the received signal to indicate on the display one or more of:
an environmental condition within the first and/or second region, and
a weight of a foodstuff within the first and/or second region.

14. A food holder according to claim 13, comprising a processor coupled to the sensor and to the display, the processor being configured to:
determine an estimated expiry date of a foodstuff within the first and/or second region in dependence on sensor data received from the sensor, and
cause the display to indicate the estimated expiry date.

15. A storage unit adapted to receive a food holder according to any preceding claim for providing separate environmental control over different regions within the food holder, the storage unit comprising:
an environmental control mechanism for communicating with a first region of a food holder received by the storage unit thereby to control an environment within the first region separately from an environment within a second region of the food holder.

16. A storage unit according to claim 15, in which the environmental control mechanism comprises a fluid outlet configured to communicate with an aperture of the food holder thereby to couple a fluid flow between the fluid outlet and an interior of the holder.

17. A storage unit according to claim 15 or claim 16, in which the environmental control mechanism comprises a fluid inlet configured to communicate with an interior of the food holder thereby to provide a return fluid flow path for fluid flow from the fluid outlet.

18. A storage unit according to claim 16 or claim 17, in which the fluid outlet of the environmental control mechanism is configured to communicate with a first aperture of the food holder and the fluid inlet of the environmental control mechanism is configured to communicate with a second aperture of the food holder, thereby to couple a fluid flow between the fluid outlet and the fluid inlet through the food holder.

19. A storage unit according to any of claims 16 to 18, in which one or both of the fluid outlet and the fluid inlet are configured for sealing engagement with respective portions of the food holder.

20. A storage unit according to any of claims 16 to 19, configured to control one or more of temperature, humidity and flow rate of fluid emitted from the fluid outlet.

21. A storage unit according to any of claims 15 to 20, comprising a first zone and a second zone, the storage unit being configured to receive the food holder such that the first region of the food holder is received into the first zone and the second region of the food holder is received into the second zone, the storage unit being configured to hold each of the first zone and the second zone at different temperatures thereby to control the temperature of the first region to be different to that of the second region.

22. A storage unit according to claim 21, in which the first zone is chilled compared to an ambient room temperature and the second zone is permitted to remain at the ambient room temperature.

23. A storage unit according to any of claims 15 to 22, comprising a reader configured to read an identifier associated with the food holder, the storage unit being configured to control the environment within the first region in dependence on the identifier.

24. A storage unit according to any of claims 15 to 23, configured to generate an indicator signal thereby to cause an indicator to display an indication of the controlled environment.

25. A food storage system for storing a plurality of foodstuffs at different environmental conditions, the food storage system comprising:
a food holder according to any of claims 1 to 14, and
a storage unit according to any of claims 15 to 24.

26. A system for determining the nutritional content of food packaging, the system comprising:
a communication module configured to receive an identifier associated with food packaging, and a processing module configured to:
access, in dependence on the received identifier, data relating to the food packaging associated with the identifier, the data being stored at a memory, the data comprising:
an indication of the type of food stored in the food packaging, and
a parameter relating to the food stored in the food packaging determined on packaging the food packaging; and
determine, in dependence on the accessed parameter, the nutritional content of the food packaging identified by the identifier.

27. A system according to claim 26, in which the parameter comprises one or more of:
a weight of the food in the food packaging,
a volume of the food in the food packaging,
an indication of the atmosphere in which the food in the food packaging was packaged, and
a date on which the food in the food packaging was packaged.

28. A system according to claim 26 or claim 27, in which the communication module is configured to receive an indication of the temperature profile of the food packaging over time and the processing module is configured to determine the nutritional content of the food packaging in dependence on the received temperature profile.

29. A system according to any of claims 26 to 28, in which the system is further configured to output a signal indicating the nutritional content of the food packaging.

30. A system according to any of claims 26 to 29, in which the communication module is configured to receive a plurality of identifiers, each identifier being associated with respective food packaging and the processing module is configured, in dependence on the respective identifier, to access data relating to the respective food packaging and determine the nutritional content of each of the plurality of food packaging.

31. A system according to any of claims 26 to 30, in which the system is further configured to output a signal indicating the nutritional content of the plurality of food packaging.

32. A system according to any of claims 26 to 31, comprising a reader for reading the identifier or the plurality of identifiers, the reader being configured to send the identifier or the plurality of identifiers to the communication module.

33. A system according to claim 32, in which the reader is remote from the memory.

34. A system according to claim 32 or claim 33, in which the reader is remote from the communication module.

35. A system according to any of claims 32 to 34, in which the memory is provided at a server remote from the reader.

36. A method of determining the nutritional content of food packaging, the method comprising:
receiving an identifier associated with food packaging,
accessing, in dependence on the received identifier, data relating to the food packaging associated with the identifier, the data comprising an indication of the type of food stored in the food packaging, and a parameter relating to the food stored in the food packaging determined on packaging, and
determining, in dependence on the accessed parameter, the nutritional content of the food packaging identified by the identifier.

37. A method according to claim 36, in which the parameter comprises one or more of:
a weight of the food in the food packaging on packing,
a volume of the food in the food packaging on packing,
an indication of the atmosphere in which the food in the food packaging was packaged, and
a date on which the food in the food packaging was packaged.

38. A method according to claim 36 or claim 37, comprising receiving an indication of the temperature profile of the food packaging over time and determining the nutritional content of the food packaging in dependence on the received temperature profile.

39. A method according to any of claims 36 to 38, comprising outputting a signal indicating the nutritional content of the food packaging.

40. A method according to any of claims 36 to 39, comprising receiving a plurality of identifiers, each identifier being associated with respective food packaging and, in dependence on the respective identifier, accessing data relating to the respective food packaging and determining the nutritional content of each of the plurality of food packaging.

41. A method according to any of claims 36 to 40, comprising outputting a signal indicating the nutritional content of the plurality of food packaging.

42. A method according to any of claims 36 to 41, comprising reading the identifier or the plurality of identifiers.

43. A method for recommending a food item from a group of food items for consumption by a user, the method comprising:
receiving user data relating to the user,
accessing food data relating to the group of food items, the food data comprising nutritional content of each of the food items in the group of food items,
determining a predicted physiological effect on the user of each food item of the group of food items in dependence on the food data and the user data, and
selecting, in dependence on the predicted physiological effect on the user, one or more food item of the group of food items for consumption by the user.

44. A method according to claim 43, in which the user data comprises one or more of:
physiological data relating to the user,
activity data relating to the user, and
data relating to food consumed by the user within a predetermined period of time preceding the selection of the food item for consumption by the user.

45. A method according to claim 44, in which the physiological data relating to the user comprises one or more of:
a height and/or weight of the user,
a fitness level of the user,
at least a portion of a DNA profile of the user,
a blood sugar level of the user,
a metabolic state of the user, and
food consumption history of the user.

46. A method according to claim 44 or claim 45, in which the physiological data comprises data based on one or more of current physiological data and previous physiological data.

47. A method according to claim 45 or claim 46, comprising determining the blood sugar level of the user in dependence on a signal from a blood sugar monitor.

48. A method according to any of claims 44 to 47, in which the activity data relating to the user comprises one or more of:
a current activity state of the user,
a previous activity state of the user, and
an expected activity state of the user.

49. A method according to any of claims 44 to 48, in which the activity data is determined in dependence on one or more of:
manually input data relating to at least one of a current activity, a previous activity and an expected activity,
a tracking device, and
a calendar entry.

50. A method according to any of claims 44 to 49, comprising determining that a change in the user's physiological data has occurred which indicates that an activity has been undertaken by the user, requesting user input relating to the indicated activity, and storing one or both of the day and time of day together with the user input, and subsequently determining the activity data in dependence on the stored data.

51. A method according to any of claims 43 to 50, comprising monitoring the user's physiological response to a particular food item, and selecting the food item for consumption by the user in dependence on the user's previous physiological response to that particular food item.

52. A method according to any of claims 43 to 51, in which the selection of the one or more food item of the group of food items for consumption by the user comprises determining a proportion of a food item for consumption by the user.

53. A method according to any of claims 43 to 52, comprising generating a signal indicative of the selected one or more food item of the group of food items for consumption by the user, thereby to cause an indicator to indicate the selected one or more food item.

54. A method according to claim 53, comprising receiving the signal at a food holder containing at least one food item and causing an indicator display of the food holder to indicate the selected one or more food item.

55. A method according to claim 53 or claim 54, comprising receiving the signal at a mobile device and causing an indicator at the mobile device to indicate the selected food item.

56. A system for recommending a food item from a group of food items for consumption by a user, the system comprising a processor configured to:
receive user data relating to the user,
access food data relating to the group of food items, the food data being stored at a memory accessible to the processor, the food data comprising nutritional content of each of the food items in the group of food items,
determine a predicted physiological effect on the user of each food item of the group of food items in dependence on the food data and the user data, and
select, in dependence on the predicted physiological effect on the user, one or more food item of the group of food items for consumption by the user.

57. A system according to claim 56, in which the user data comprises one or more of:
physiological data relating to the user,
activity data relating to the user, and
data relating to food consumed by the user within a predetermined period of time preceding the selection of the food item for consumption by the user.

58. A system according to claim 57, in which the physiological data relating to the user comprises one or more of:
a height and/or weight of the user,
a fitness level of the user,
at least a portion of a DNA profile of the user,
a blood sugar level of the user,
a metabolic state of the user, and
food consumption history of the user.

59. A system according to claim 57 or claim 58, in which the physiological data comprises data based on one or more of current physiological data and previous physiological data.

60. A system according to claim 58 or claim 59, in which the blood sugar level of the user is determined in dependence on a blood sugar monitor.

61. A system according to any of claims 57 to 60, in which the activity data relating to the user comprises one or more of:
a current activity state of the user,
previous activity state of the user, and
an expected activity state of the user.

62. A system according to any of claims 57 to 61, in which the activity data is determined in dependence on one or more of:
manually input data relating to at least one of a current activity, a previous activity and an expected activity,
a tracking device, and
a calendar entry.

63. A system according to any of claims 57 to 62, in which the processor is further configured to:
determine that a change in the user's physiological data has occurred which indicates that an activity has been undertaken by the user,
request user input relating to the indicated activity,
store one or both of the day and time of day together with the user input, and
determine the activity data in dependence on the stored data.

64. A system according to any of claims 56 to 63, in which the processor is further configured to:
monitor the user's physiological response to a particular food item, and
select the food item for consumption by the user in dependence on the user's previous physiological response to that particular food item.

65. A system according to any of claims 56 to 64, in which the processor is configured to select the one or more food item of the group of food items for consumption by the user by determining a proportion of a food item for consumption by the user.

66. A system according to any of claims 56 to 65, in which the processor is configured to generate a signal indicative of the selected one or more food item of the group of food items for consumption by the user, thereby to cause the display of the selected one or more food item.

67. A system according to claim 66, comprising a food holder containing at least one food item, the food holder being configured to receive the signal and cause an indicator display of the food holder to indicate the selected one or more food item.

68. A system according to claim 66 or claim 67, comprising a mobile device configured to receive the signal and cause an indicator at the mobile device to indicate the determined food item.

69. Food packaging comprising a tag configured to transmit a signal indicative of the preparation of the food packaging for consumption of food contained in the food packaging.

70. Food packaging according to claim 69, in which the tag is configured to detect an indication of the preparation of the food packaging.

71. Food packaging according to claim 69 or claim 70, in which the tag comprises a sensor configured to detect the separation of one portion of the food packaging from another portion of the food packaging and to generate a separation signal in dependence on the detected separation, the signal indicative of the preparation comprising the separation signal.

72. Food packaging according to claim 71, in which the tag is configured to transmit the signal indicative of the preparation in dependence on the detection of the separation.

73. Food packaging according to claim 71 or claim 72, in which the tag comprises a proximity sensor configured to detect the separation and to generate the separation signal in dependence on the detected separation.

74. Food packaging according to any of claims 71 to 73, in which the tag comprises one or both of a magnetic sensor and a capacitive sensor for detecting the separation.

75. Food packaging according to any of claims 71 to 74, comprising a cover and a body, the tag being configured to detect the separation of a portion of the cover from the body and to generate the separation signal in dependence on determining that the portion of the cover is separated from the body.

76. Food packaging according to any of claims 71 to 75, in which the tag is configured to detect the separation in dependence on determining a change in an electrically conductive path through at least a portion of the food packaging.

77. Food packaging according to any of claims 69 to 76, in which the tag is configured to detect a temperature of the food packaging and to generate a readiness signal in dependence on the temperature, the signal indicative of the preparation comprising the readiness signal.

78. Food packaging according to claim 77, in which the tag is configured to transmit the signal indicative of the preparation in dependence on the readiness signal.

79. Food packaging according to claim 77 or claim 78, in which the tag is configured to generate the readiness signal in dependence on the temperature satisfying a temperature criterion.

80. Food packaging according to any of claims 77 to 79, in which the tag is configured to detect a temperature profile of the food packaging over time, and to determine that the temperature satisfies the temperature criterion in dependence on the detected temperature profile.

81. Food packaging according to claim 79 or claim 80, in which the temperature criterion comprises a threshold temperature, and the tag is configured to determine that the temperature criterion is satisfied in dependence on the temperature exceeding the threshold temperature.

82. Food packaging according to any of claims 79 to 81, in which the temperature criterion comprises a threshold thermal budget, and the tag is configured to determine that the temperature criterion is satisfied in dependence on the temperature profile indicating that the thermal budget of the food packaging exceeds the threshold thermal budget.

83. Food packaging according to any of claims 79 to 82, in which the temperature criterion comprises a further threshold temperature, and the tag is configured to determine that the temperature criterion is satisfied in dependence on one or both of:
the temperature exceeding the threshold temperature, and
the thermal budget of the food packaging exceeding the threshold thermal budget, and in dependence on the temperature subsequently falling below the further threshold temperature.

84. Food packaging according to any of claims 69 to 83, in which the tag is configured to determine movement of the food packaging and to transmit the signal indicative of the preparation in dependence on the determined movement.

85. Food packaging according to claim 84, in which the tag is configured to determine movement of the food packaging in dependence on a weight signal generated by a weight sensor.

86. Food packaging according to claim 84 or claim 85, in which the tag is configured to determine a distance from a location external to the food packaging and to determine movement of the food packaging in dependence on the determined distance.

87. Food packaging according to claim 86, in which the tag is configured to determine the distance in dependence on a received signal strength of a signal generated at the external location.

88. Food packaging according to any of claims 69 to 87, in which the signal indicative of the preparation of the food packaging identifies the food contained in the food packaging.

89. A method for detecting the readiness for use of food packaging, the method comprising:
receiving a signal transmitted by a tag associated with the food packaging, the signal indicating preparation of the food packaging for consumption of food contained in the food packaging, and
determining readiness for use of the food packaging in dependence on the received signal.

90. A method according to claim 89, comprising:
determining, in dependence on the received signal, a separation between one portion of the food packaging and another portion of the food packaging, and
determining the readiness for use in dependence on the determined separation.

91. A method according to claim 89 or claim 90, comprising:
determining, in dependence on the received signal, a temperature of the food packaging, and
determining the readiness for use in dependence on the determined temperature.

92. A method according to claim 91, comprising:
determining that the temperature exceeds a threshold temperature and determining the readiness for use in dependence thereon.

93. A method according to claim 91 or claim 92, comprising:
determining a thermal budget of the food packaging based on the determined temperature;
determining that the thermal budget exceeds a threshold thermal budget and determining the readiness for use in dependence thereon.

94. A method according to any of claims 91 to 93, comprising:
determining the readiness for use in dependence on:
determining that the temperature exceeds a threshold temperature, and/or determining that a thermal budget of the food packaging exceeds a threshold thermal budget; and
determining that the temperature subsequently falls below a further threshold temperature.

95. A method according to any of claims 89 to 94, comprising determining movement of the food packaging and determining readiness for use in dependence on the determined movement.

96. A method according to claim 95, comprising receiving a weight signal generated by a weight sensor and determining movement in dependence thereon.

97. A method according to claim 95 or claim 96, comprising determining a distance between the food packaging and a location external to the food packaging, and determining movement in dependence thereon.
